SEQUENCE LISTING (1 sequence)

SEQ ID NO: 1 - protein, 2944 aa, Homo sapiens
MTLRLLVAALCAGILAEAPRVRAQHRERVTCTRLYAADIVFLLDGSSSIGRSNFREVRSFLEGLVLPFSGAASAQGVRFATVQYSDDPRTEFGLDALGSGGDVIRAIRELSYKGGNTRTGAAILHVADHVFLPQLARPGVPKVCILITDGKSQDLVDTAAQRLKGQGVKLFAVGIKNADPEELKRVASQPTSDFFFFVNDFSILRTLLPLVSRRVCTTAGGVPVTRPPDDSTSAPRDLVLSEPSSQSLRVQWTAASGPVTGYKVQYTPLTGLGQPLPSERQEVNVPAGETSVRLRGLRPLTEYQVTVIALYANSIGEAVSGTARTTALEGPELTIQNTTAHSLLVAWRSVPGATGYRVTWRVLSGGPTQQQELGPGQGSVLLRDLEPGTDYEVTVSTLFGRSVGPATSLMARTDASVEQTLRPVILGPTSILLSWNLVPEARGYRLEWRRETGLEPPQKVVLPSDVTRYQLDGLQPGTEYRLTLYTLLEGHEVATPATVVPTGPELPVSPVTDLQATELPGQRVRVSWSPVPGATQYRIIVRSTQGVERTLVLPGSQTAFDLDDVQAGLSYTVRVSARVGPREGSASVLTVRREPETPLAVPGLRVVVSDATRVRVAWGPVPGASGFRISWSTGSGPESSQTLPPDSTATDITGLQPGTTYQVAVSVLRGREEGPAAVIVARTDPLGPVRTVHVTQASSSSVTITWTRVPGATGYRVSWHSAHGPEKSQLVSGEATVAELDGLEPDTEYTVHVRAHVAGVDGPPASVVVRTAPEPVGRVSRLQILNASSDVLRITWVGVTGATAYRLAWGRSEGGPMRHQILPGNTDSAEIRGLEGGVSYSVRVTALVGDREGTPVSIVVTTPPEAPPALGTLHVVQRGEHSLRLRWEPVPRAQGFLLHWQPEGGQEQSRVLGPELSSYHLDGLEPATQYRVRLSVLGPAGEGPSAEVTARTESPRVPSIELRVVDTSIDSVTLAWTPVSRASSYILSWRPLRGPGQEVPGSPQTLPGISSSQRVTGLEPGVSYIFSLTPVLDGVRGPEASVTQTPVCPRGLADVVFLPHATQDNAHRAEATRRVLERLVLALGPLGPQAVQVGLLSYSHRPSPLFPLNGSHDLGIILQRIRDMPYMDPSGNNLGTAVVTAHRYMLAPDAPGRRQHVPGVMVLLVDEPLRGDIFSPIREAQASGLNVVMLGMAGADPEQLRRLAPGMDSVQTFFAVDDGPSLDQAVSGLATALCQASFTTQPRPEPCPVYCPKGQKGEPGEMGLRGQVGPPGDPGLPGRTGAPGPQGPPGSATAKGERGFPGADGRPGSPGRAGNPGTPGAPGLKGSPGLPGPRGDPGERGPRGPKGEPGAPGQVIGGEGPGLPGRKGDPGPSGPPGPRGPLGDPGPRGPPGLPGTAMKGDKGDRGERGPPGPGEGGIAPGEPGLPGLPGSPGPQGPVGPPGKKGEKGDSEDGAPGLPGQPGSPGEQGPRGPPGAIGPKGDRGFPGPLGEAGEKGERGPPGPAGSRGLPGVAGRPGAKGPEGPPGPTGRQGEKGEPGRPGDPAVVGPAVAGPKGEKGDVGPAGPRGATGVQGERGPPGLVLPGDPGPKGDPGDRGPIGLTGRAGPPGDSGPPGEKGDPGRPGPPGPVGPRGRDGEVGEKGDEGPPGDPGLPGKAGERGLRGAPGVRGPVGEKGDQGDPGEDGRNGSPGSSGPKGDRGEPGPPGPPGRLVDTGPGAREKGEPGDRGQEGPRGPKGDPGLPGAPGERGIEGFRGPPGPQGDPGVRGPAGEKGDRGPPGLDGRSGLDGKPGAAGPSGPNGAAGKAGDPGRDGLPGLRGEQGLPGPSGPPGLPGKPGEDGKPGLNGKNGEPGDPGEDGRKGEKGDSGASGREGRDGPKGERGAPGILGPQGPPGLPGPVGPPGQGFPGVPGGTGPKGDRGETGSKGEQGLPGERGLRGEPGSVPNVDRLLETAGIKASALREIVETWDESSGSFLPVPERRRGPKGDSGEQGPPGKEGPIGFPGERGLKGDRGDPGPQGPPGLALGERGPPGPSGLAGEPGKPGIPGLPGRAGGVGEAGRPGERGERGEKGERGEQGRDGPPGLPGTPGPPGPPGPKVSVDEPGPGLSGEQGPPGLKGAKGEPGSNGDQGPKGDRGVPGIKGDRGEPGPRGQDGNPGLPGERGMAGPEGKPGLQGPRGPPGPVGGHGDPGPPGAPGLAGPAGPQGPSGLKGEPGETGPPGRGLTGPTGAVGLPGPPGPSGLVGPQGSPGLPGQVGETGKPGAPGRDGASGKDGDRGSPGVPGSPGLPGPVGPKGEPGPTGAPGQAVVGLPGAKGEKGAPGGLAGDLVGEPGAKGDRGLPGPRGEKGEAGRAGEPGDPGEDGQKGAPGPKGFKGDPGVGVPGSPGPPGPPGVKGDLGLPGLPGAPGVVGFPGQTGPRGEMGQPGPSGERGLAGPPGREGIPGPLGPPGPPGSVGPPGASGLKGDKGDPGVGLPGPRGERGEPGIRGEDGRPGQEGPRGLTGPPGSRGERGEKGDVGSAGLKGDKGDSAVILGPPGPRGAKGDMGERGPRGLDGDKGPRGDNGDPGDKGSKGEPGDKGSAGLPGLRGLLGPQGQPGAAGIPGDPGSPGKDGVPGIRGEKGDVGFMGPRGLKGERGVKGACGLDGEKGDKGEAGPPGRPGLAGHKGEMGEPGVPGQSGAPGKEGLIGPKGDRGFDGQPGPKGDQGEKGERGTPGIGGFPGPSGNDGSAGPPGPPGSVGPRGPEGLQGQKGERGPPGERVVGAPGVPGAPGERGEQGRPGPAGPRGEKGEAALTEDDIRGFVRQEMSQHCACQGQFIASGSRPLPSYAADTAGSQLHAVPVLRVSHAEEEERVPPEDDEYSEYSEYSVEEYQDPEAPWDSDDPCSLPLDEGSCTAYTLRWYHRAVTGSTEACHPFVYGGCGGNANRFGTREACERRCPPRVVQSQGTGTAQD

(19) United States
(12) Patent Application Publication
(10) Pub. No.: US 2019/0298809 A1
(43) Pub. Date: Oct. 3, 2019

(54) RECOMBINANT C7 AND METHODS OF USE

(71) Applicants: UNIVERSITY OF SOUTHERN CALIFORNIA, Los Angeles, CA (US); PHOENIX TISSUE REPAIR, INC., Boston, MA (US)

(72) Inventors: Mei Chen, Altadena, CA (US); David Woodley, Altadena, CA (US); Mark de Souza, Boston, MA (US)

(21) Appl. No.: 16/412,192

(22) Filed: May 14, 2019

Related U.S. Application Data
(63) Continuation of application No. 13/946,847, filed on Jul. 19, 2013, now abandoned.
(60) Provisional application No. 61/673,657, filed on Jul. 19, 2012.

(51) Int. Cl. A61K 38/39

(52) U.S. Cl. CPC A61K 38/39

(57) ABSTRACT

The present disclosure provides methods of treating epidermolysis bullosa, and/or preventing, preventing the progression of, or delaying the onset of one or more symptom associated with scarring, e.g., of blisters, in subjects with epidermolysis bullosa through chronic systemic administration of collagen 7.

### CROSS-REFERENCE TO RELATED APPLICATIONS

This application is a continuation of U.S. patent application Ser. No. 13/946,847; which claims the benefit of U.S. Provisional Application No. 61/673,657, filed Jul. 19, 2012, the contents of which are hereby incorporated by reference in their entirety.

### GOVERNMENT SUPPORT

This invention was made with government support under R01 AR033625 and R01 AR047981 awarded by the National Institutes of Health. The government has certain rights in the invention.

### BACKGROUND OF THE INVENTION

Epidermolysis bullosa (EB) is a group of genetic conditions that cause the skin to be very fragile and to blister easily. Blisters and skin erosions form in response to minor injury or friction, such as rubbing or scratching. Dystrophic epidermolysis bullosa (DEB) is one of the major forms of epidermolysis bullosa. The signs and symptoms of this condition vary widely among affected individuals. In mild cases, blistering may primarily affect the hands, feet, knees, and elbows. Severe cases of this condition involve widespread blistering that can lead to vision loss, disfigurement, and other serious medical problems.

Researchers classify dystrophic epidermolysis bullosa into three major types. Although the types differ in severity, their features overlap significantly and they are all caused by mutations in the COL7A1 gene, which encodes for the protein collagen 7. Collagen 7 functions to strengthen and stabilize the skin, and is a major component of anchoring fibrils (“AFs”), which help anchor the top layer of the skin, the epidermis, to the underlying dermis.

Recessive dystrophic epidermolysis bullosa (RDEB), the most severe and classical form of the disease, is characterized by extensive blistering and scarring of the skin and mucosal membranes. The COL7A1 mutations associated with RDEB impair the ability of collagen 7 to connect the epidermis and dermis; and subsequent separation of the epidermis and dermis as a result of friction or minor injury causes the severe blistering and extensive scarring of the skin associated with RDEB. The onset of disease symptoms is usually at birth or early infancy, with generalized blistering presenting at birth. Subsequent extensive dystrophic scarring, most prominent on the acral surfaces, often leads to the development of pseudosyndactyly of the hands (i.e. mitten-hand deformity) and the feet during infancy. Flexion contractures of the extremities also become increasingly common with age, along with rectal and esophageal blistering.

A second type of autosomal recessive dystrophic epidermolysis bullosa is known as the non-Hallopeau-Siemens type (non-HS RDEB). This form of the condition is somewhat less severe than the classic type and includes a range of subtypes. Blistering is limited to the hands, feet, knees, and elbows in mild cases, but may be widespread in more severe cases. Affected people often have malformed fingernails and toenails. Non-HS RDEB involves scarring in the areas where blisters occur, but this form of the condition does not cause the severe scarring characteristic of the classic type.

The third major type of dystrophic epidermolysis bullosa is known as the autosomal dominant type (DDEB). The signs and symptoms of this condition tend to be milder than those of the autosomal recessive forms, with blistering often limited to the hands, feet, knees, and elbows. The blisters heal with scarring, but it is less severe. Most affected people have malformed fingernails and toenails, and the nails may be lost over time. In the mildest cases, abnormal nails are the only sign of the condition.

### SUMMARY OF THE INVENTION

The present disclosure is based, at least in part, on the finding that administration of collagen 7, and functional fragments thereof, e.g., by systemic administration, e.g., intravenous administration, homes not only to wounded skin but also unwounded skin and other squamous epithelial tissue, e.g., in subjects with epidermolysis bullosa (EB), e.g., dystrophic epidermolysis bullosa (DEB), e.g., recessive dystrophic epidermolysis bullosa (RDEB) or dominant dystrophic epidermolysis (DDEB). Accordingly, the disclosure provides, inter alia, methods of treating EB, (e.g., DEB, e.g., RDEB or DDEB) and/or preventing, preventing the progression of, or delaying the onset of one or more symptom associated with scarring, e.g., scarring of blisters, in subjects with EB (e.g., DEB, e.g., RDEB or DDEB) through chronic administration of collagen 7.

In one aspect, the disclosure features a method of treating EB, e.g., DEB, e.g., RDEB or DDEB, the method comprising: systemically, e.g., intravenously, administering to a subject having EB (e.g., DEB, e.g., RDEB or DDEB) or at risk of having EB (e.g., DEB, e.g., RDEB or DDEB) collagen 7, or functional fragments and variants thereof, and administering subsequent doses of collagen 7, or the functional fragment or variant thereof, such that the subject is administered collagen 7, or the functional fragment or variant thereof, over a period of sufficient frequency and dosage to maintain an effective amount of C7 in the skin and other squamous epithelial tissue beyond the healing of lesions present at the time of initial administration, e.g., for a period sufficient to treat chronic or long term symptoms associated with EB, e.g., DEB, e.g., RDEB or DDEB. For example, the period is sufficient to treat chronic or long term symptoms associated with scarring, e.g., from blisters associated with EB, e.g., DEB, e.g., RDEB or DDEB.

In one embodiment, the collagen 7, or the functional fragment or variant thereof, is administered for a period of at least 3 months, 4 months, 5 months, 6 months, 9 months, 12 months, 15 months, 18 months, 21 months, 2 years, three years, fours years, five years, six years or more (e.g., over a lifetime).

In one embodiment, each subsequent dose is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, collagen 7 is administered intravenously.

In one embodiment, the subject is less than 24 months of age, less than 12 months of age, less than 6 months of age, less than 5 months of age, less than 4 months of age, less than 3 months of age, less than 2 months of age, or less than 1 month of age.

In one embodiment, the subject is at risk of EB, e.g., DEB, e.g., RDEB or DDEB. In one embodiment, the subject has a mutation in the COL7A1 gene associated with EB, e.g., DEB, e.g., RDEB, e.g., the subject has a mutation in one or more of exons 73, 74, and 75 of the COL7A1 gene. It should be appreciated the references to the one or more exons of 73, 74 and 75 is only one example and mutations outside these exons may also occur.

In one aspect, the disclosure features a method of preventing, preventing the progression of, or delaying the onset of one or more symptom associated with scarring, e.g., scarring of blisters, in subjects with EB (e.g., DEB, e.g., RDEB or DDEB). The method comprises systemically administering, e.g., by intravenous administration, collagen 7, or a functional fragment or variant thereof, to a subject having EB (e.g., DEB, e.g., RDEB or DDEB), or at risk of having EB (e.g., DEB, e.g., RDEB or DDEB), and administering subsequent doses of collagen 7, or the functional fragment or variant thereof, such that the subject is administered collagen 7, or the functional fragment or variant thereof, over a period of sufficient frequency and dosage to maintain an effective amount of C7 beyond the healing of lesions present at the time of initial administration.

In one embodiment, the collagen 7, or the functional fragment or variant thereof, is administered for a period of at least 3 months, 4 months, 5 months, 6 months, 9 months, 12 months, 15 months, 18 months, 21 months, 2 years, three years, four years, five years, six years or more (e.g., over a lifetime).

In one embodiment, each subsequent dose is administered two weeks, three weeks, four weeks, five weeks, six weeks, eight weeks after the previous dose. In one embodiment, each subsequent dose is administered one month after the previous dose of collagen 7, or the functional fragment or variant thereof.

In one embodiment, the collagen 7, or functional fragment or variant thereof, is administered once a month for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 6 weeks, for at least 3 months, e.g., at least 4, 5, 6, 9, 12, 15, 18, 24, 27, 30, 33, 36, 39, 42, 45 or 48 months or more.

In one embodiment, the collagen 7, or functional fragment or variant thereof, is administered once every 2 months, for at least 3 months, e.g., at least 4, 5, 6, 9, 12, 15, 18, 24, 27, 30, 33, 36, 39, 42, 45 or 48 months or more.

In one embodiment, collagen 7 is administered intravenously.

In one embodiment, the subject is less than 24 months of age, less than 12 months of age, less than 6 months of age, less than 5 months of age, less than 4 months of age, less than 3 months of age, less than 2 months of age, or less than 1 month of age.

In one embodiment, the subject is at risk of EB, e.g., DEB, e.g., RDEB or DDEB. In one embodiment, the subject has a mutation in the COL7A1 gene associated with EB, e.g., DEB, e.g., RDEB, e.g., the subject has a mutation in one or more of exons 73, 74, and 75 of the COL7A1 gene.

In one embodiment, the symptom associated with scarring is: contractures, e.g., flexion contractures (e.g., of the extremities); pseudosyndactyly, e.g., pseudosyndactyly of the hands and pseudosyndactyly of the feet; carcinoma (e.g., squamous cell carcinoma); rectal lesions; urethral lesions; anal lesions; mucosal lesions; lesions of squamous epithelial tissue; lesions of the gastrointestinal tract; bulla formation; bulla formation post manual trauma; nail deformities; teeth deformities; constricted esophagus; eye disorders, anemia, malnutrition; secondary skin infection; sepsis; hoarse voice; urethral stenosis; phimosis; corneal scarring; malabsorption; and failure to thrive.

### BRIEF DESCRIPTION OF THE FIGURES

FIG. 1 immunofluorescence shows systemically administered collagen 7 homes to skin wounds, and specifically the basement membrane zone of the wounded skin in RDEB mice.

FIG. 2 immunofluorescence shows systemically administered collagen 7 homes to wounded skin in a dose dependent manner in RDEB mice.

FIG. 3 immunofluorescence shows systemically administered collagen 7 homes to the wounded area of the tongue in RDEB mice.

FIG. 4 immunofluorescence shows chronically administered collagen 7 homes to the skin, tongue, and esophagus in RDEB mice.

FIG. 5 shows chronic administration of collagen 7 delays forepaw pseudosyndactyly in RDEB mice.

FIG. 6 immunofluorescence analysis shows the deposition of collagen 7 in the high stress areas of the digit dermal-epidermal junction of the forepaws of RDEB mice chronically administered collagen 7.

FIG. 7 shows chronic administration of collagen 7 protects against rectal blistering in RDEB mice.

FIG. 8 shows chronic administration of collagen 7 results in a highly decreased percentage of rectal blistering 6 weeks post the initiation of collagen 7 treatment in RDEB mice.

FIG. 9 is a schematic representation of the athymic nude mouse model utilized to evaluate the homing and deposition of intravenously administered recombinant collagen 7. In brief, skin from collagen 7 knockout DEB mice is grafted onto athymic nude mice. After two weeks, the DEB skin is wounded by 6mm punch biopsies or left unwounded and recombinant collagen 7 administered IV via tail vein injection. The mice are then sacrificed at two to three weeks post collagen 7 administration, and skin biopsies from the grafted DEB skin analyzed by hematoxylin and eosin stain, immunofluorescence, or electron microscopy.

FIG. 10A shows unwounded or 6mm punch biopsy wounded athymic nude mice 2 weeks post grafting of collagen 7 knockout DEB skin grafts. FIG. 10B shows immunofluorescence staining conducted two weeks post punch biopsy wounding of athymic nude mice with collagen 7 knockout DEB skin grafts, using the anti-collagen 7 NC1 domain pAb, and shows no collagen 7 expression. FIG. 10C shows hematoxylin and eosin staining conducted two weeks post punch biopsy wounding of athymic nude mice with collagen 7 knockout DEB skin grafts and immunofluorence staining of cysections using the anti-collagen 7 NC1 domain pAb. The engrafted RDEB skin shows no collagen 7 expression and dermal-epidermal separation. These data indicated that knockout DEB skin retained RDEB disease features.

FIG. 11A shows immunofluorescence staining conducted two to three weeks post wounding of collagen 7 knockout DEB skin grafts on athymic nude micereceived30-120 μg IV administration of recombinant collagen 7. Immunofluorescence staining was conducted, using the anti-human collagen 7 mAb (LH7.2), and showed the restoration of collagen 7 expression in the basement membrane zone of the collagen 7 knockout DEB skin grafts. In contrast, DEB skin grafts received IV PBS had not collagen 7 expression at the BMZ. FIG. 11B shows immunofluorescence staining conducted two to three weeks post wounding of collagen 7 knockout DEB skin grafts on athymic nude mice received 30-120 μg IV administration of recombinant collagen 7. Immunofluorescence staining was conducted, using the anti-collagen 7 NC1 domain pAb, and showed the restoration of collagen 7 expression in the basement membrane zone of the collagen 7 knockout DEB skin grafts.

FIG. 12 shows hematoxylin and eosin staining conducted two to three weeks post wounding of collagen 7 knockout DEB skin grafts on athymic nude mice received 30-120 μg IV administration of recombinant collagen 7, and showed the correction of the dermal-epidermal separation seen in the collagen 7 knock out wounded skin grafts by the IV administration collagen 7. In contrast, DEB skin grafts IV injected with PBS exhibited dermal-epidermal separation.

FIG. 13 shows immunofluorescence staining conducted two to three weeks post wounding of collagen 7 knockout DEB skin grafts on athymic nude mice received 30 and 60 μg IV administration of recombinant collagen 7, and shows a dose dependent deposition of recombinant collagen 7 at the basement membrane zone of the wounded skin grafts. Immunofluorescence staining was conducted using the anti-collagen 7 NC1 domain pAb.

FIG. 14A shows immunofluorescence staining conducted one to two weeks post 60 μg IV administration of recombinant collagen 7 to unwounded collagen 7 knockout DEB skin grafted onto athymic nude mice. Immunofluorescence staining was conducted, using anti-human collagen 7 mAb (LH7.2), and showed the restoration of collagen 7 expression at the basement membrane zone of the unwounded skin. FIG. 14B shows hematoxylin and eosin staining conducted one to two weeks post 60 μg IV administration of recombinant collagen 7 to collagen 7 knockout DEB skin grafted onto athymic nude mice without wounding Note that IV C7 also homed to unwounded DEB skin grafts and corrected dermal-epidermal separation.

FIG. 15 shows immunofluorescence staining conducted two to three weeks post IV administration of 60-120 μg of recombinant collagen 7 to unwounded athymic nude mice with collagen 7 knockout DEB skin grafts. All organ biopsies were obtained from unwounded mice except, a skin graft obtained from wounded athymic nude mice with collagen 7 knockout for skin grafts for analysis in the panel labeled ‘wounded KO skin’. Immunofluorescence staining was conducted, using anti-human collagen 7 mAb (LH7.2), and showed that IV injected human C7 did not traffick to other internal organs and unwounded skin of athymic nude mice except of wounded DEB skin grafts.

### DETAILED DESCRIPTION OF THE INVENTION

Certain terms are first defined. Additional terms are defined throughout the specification.

“Chronic administration”, as used herein, refers to the administration of more than one dose of an agent over a period of time. Chronic administration can include regular administration for an extended period of time. Chronic administration can also include the administration of therapy over a prolonged period of time (in some cases, for the duration of a subject's lifetime) so that the concentration of the therapeutic agent is maintained at a therapeutically or prophylactically effective level throughout the course of treatment.

An “effective amount” of collagen 7 or functional fragment or variant thereof refers to the amount of collagen 7 or functional fragment or variant thereof, when administered in an accumulate of multiple doses, or as part of any other type of defined treatment regimen, produces a measureable statistical improvement in outcome, as evidenced by at least one clinical parameter associated with the complication.

“Recombinant”, as used herein, in reference to a protein or polypeptide molecule, pertains to a protein or polypeptide molecule expressed utilizing isolated nucleic acid molecules or recombinant nucleic acid molecules.

“Collagen 7” as used herein refers to collagen type 7 encoded by the COL7A1 gene. Collagen 7 consists of 2,944 amino acids. It comprises a non-collagenous NC1 domain (including residues 17-1253 in the mature peptide), the central collagenous helical domain (residues 1254-2783), and the carboxyl-terminal NC2 domain (residues 2784-2944).

A functional fragment of collagen 7 refers to a portion of collagen 7 that maintains the ability to form anchoring fibrils between the epidermal and dermal layers of human skin, and the ability to bind collagen 4 and laminin-332. For example, a functional fragment can include all or a portion of the NC1 domain and/or the NC2 domain of collagen 7, e.g., the functional fragment can be collagen 7 without all of a portion of the central collagenous helical domain, e.g., a fragment that does not include amino acid residues 1920-2603 of the central collagenous helical domain of collagen 7.

A variant of collagen 7 refers to a polypeptide that has substantial identity with collagen 7 that maintains the ability to form anchoring fibrils between the epidermal and dermal layers of human skin. Collagen 7 variants include, but are not limited to, collagen 7 polypeptides that have been either chemically modified relative to collagen 7 and/or contain one or more amino acid sequence alterations relative to collagen 7.

Variants of collagen 7 include polypeptides having at least 75%, 80%, 85%, 90%, 95%, 96%, 97%, 98% or 99% identity with the amino acid sequence of human collagen 7 (see infra). Calculations of “identity” or “sequence homology” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

Variants of collagen 7 also include polypeptides having amino acid modifications (e.g., deletions, additions or substitutions, such as conservative substations) from the amino acid sequence of collagen 7 (See infra). For example, a variant of collagen 7 can differ by at least 1, 2, 3, 4, 5 but not more than 50, 40, 30, 20, 15 or 10 amino acids from collagen 7 (see infra). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

“Collagen 7 composition” refers to a plurality of collagen 7 polypeptides or collagen 7 equivalent polypeptides, or functional fragments or variants of collagen 7, including variants and chemically modified forms that have been separated from the cell in which they were synthesized. An “isolated composition” refers to a composition that is removed from at least 90% of at least one component of a natural sample from which the isolated composition can be obtained. Compositions produced artificially or naturally can be “compositions of at least” a certain degree of purity if the species or population of species of interests is at least 5, 10, 25, 50, 75, 80, 90, 92, 95, 98, or 99% pure on a weight-weight basis.

An “isolated” protein refers to a protein that is removed from at least 90% of at least one component of a natural sample from which the isolated protein can be obtained. Proteins can be “of at least” a certain degree of purity if the species or population of species of interest is at least 5, 10, 25, 50, 75, 80, 90, 92, 95, 98, or 99% pure on a weight-weight basis.

The term “preventing” a disease in a subject refers to subjecting the subject to a pharmaceutical treatment, e.g., the administration of a drug, such that at least one symptom of the disease is prevented, that is, administered prior to clinical manifestation of the unwanted condition (e.g., disease or other unwanted state of the host animal) so that it protects the host against developing the unwanted condition. “Preventing” a disease may also be referred to as “prophylaxis” or “prophylactic treatment.” In the present disclosure, one or more symptom of scarring, e.g., associated with blistering and/or lesions in subjects with EB (e.g., DEB, e.g., RDEB or DDEB), can be prevented. For example, scarring in subjects with EB (e.g., DEB, e.g., RDEB or DDEB) can result in one or more of the following symptoms: contractures, e.g., flexion contractures (e.g., of the extremities); pseudosyndactyly, e.g., pseudosyndactyly of the hands and pseudosyndactyly of the feet; carcinoma (e.g., squamous cell carcinoma); rectal lesions; mucosal lesions; bulla formation; bulla formation post manual trauma; nail deformities; teeth deformities; constricted esophagus; eye disorders, anemia, malnutrition; secondary skin infection; sepsis; hoarse voice; urethral stenosis; phimosis; corneal scarring; malabsorption; and failure to thrive.

“Treating” EB, e.g., DEB, e.g., RDEB or DDEB, in a subject or “treating” a subject having EB, e.g., DEB, e.g., RDEB or DDEB, refers to subjecting the subject to a pharmaceutical treatment, e.g., the administration of a drug, such that at least one symptom of the disease is cured, alleviated or decreased. “Treating” a EB may be evaluated by any one of the following parameters: blistering; lesions (e.g., rectal, anal, urethral lesions and/or mucosal lesions and/or lesions of squamous epithelial tissue); lesions of the gastrointestinal tract; contractures, e.g., flexion contractures (e.g., of the extremities); pseudosyndactyly, e.g., pseudosyndactyly of the hands and pseudosyndactyly of the feet; carcinoma (e.g., squamous cell carcinoma); bulla formation; nail deformities; teeth deformities; constricted esophagus; eye disorders, anemia, malnutrition; secondary skin infection; sepsis; hoarse voice; urethral stenosis; phimosis; corneal scarring; malabsorption; and failure to thrive.

A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein to elicit a desired response in the individual. A therapeutically effective amount is also one in which any toxic or detrimental effects of the composition are outweighed by the therapeutically beneficial effects.

A “patient”, “subject” or “host” (these terms are used interchangeably) to be treated by the subject method may mean either a human or non-human animal.

Any of the treatments described herein can be administered in combination with another agent or therapy. The term “combination” refers to the use of the two or more agents or therapies to treat the same patient, wherein the use or action of the agents or therapies overlap in time. The agents or therapies can be administered at the same time (e.g., as a single formulation that is administered to a patient or as two separate formulations administered concurrently) or sequentially in any order.

### Epidermolysis Bullosa (EB)

Epidermolysis bullosa is a group of inherited genetic conditions that cause the skin to be very fragile and to blister easily. Blisters and skin erosions form spontaneously and in response to minor injury or friction, such as rubbing or scratching. The condition generally starts at birth or soon after. Dystrophic epidermolysis bullosa (DEB) is one of the major forms of epidermolysis bullosa. The signs and symptoms of this condition vary widely among affected individuals. In mild cases, blistering may primarily affect the hands, feet, knees, and elbows. Severe cases of this condition involve widespread blistering and scarring that can lead to vision loss, disfigurement, and other serious medical problems.

The term “unwounded” when used in connection with skin or other squamous epithelial tissue means that there are no visible wounds in the tissue of interest when viewed with the naked eye. It should be understood that the unwounded skin of a subject with EB may be different from the unwounded skin of a subject without EB. Specifically, at least a portion of the unwounded skin, or other squamous epithelial tissue, in a patient with EB may be characterized by sufficiently low levels of C7 to cause, for example subepidermal wounds, tissue separations or other adverse effects that do not result in visible wounds.

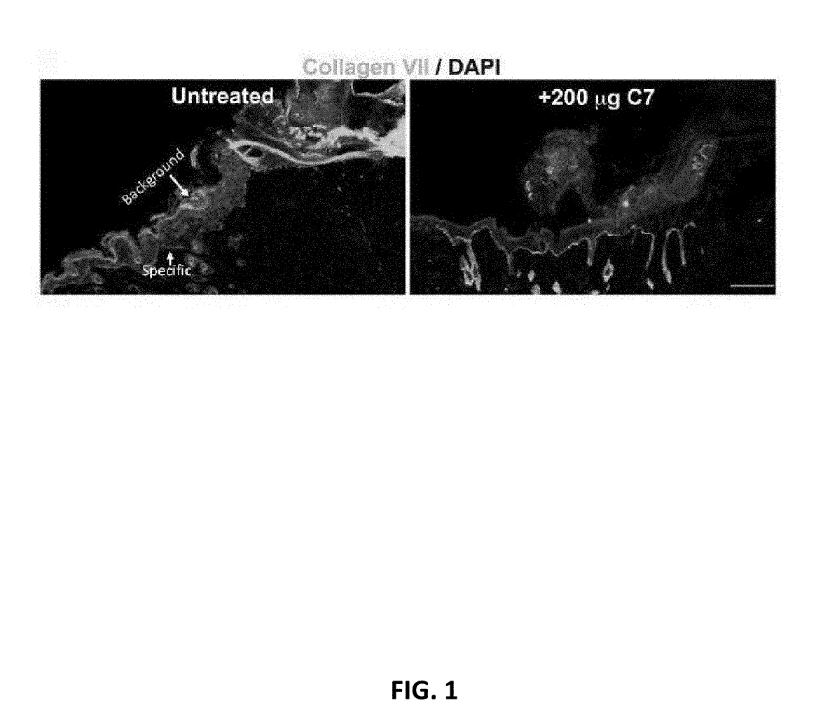

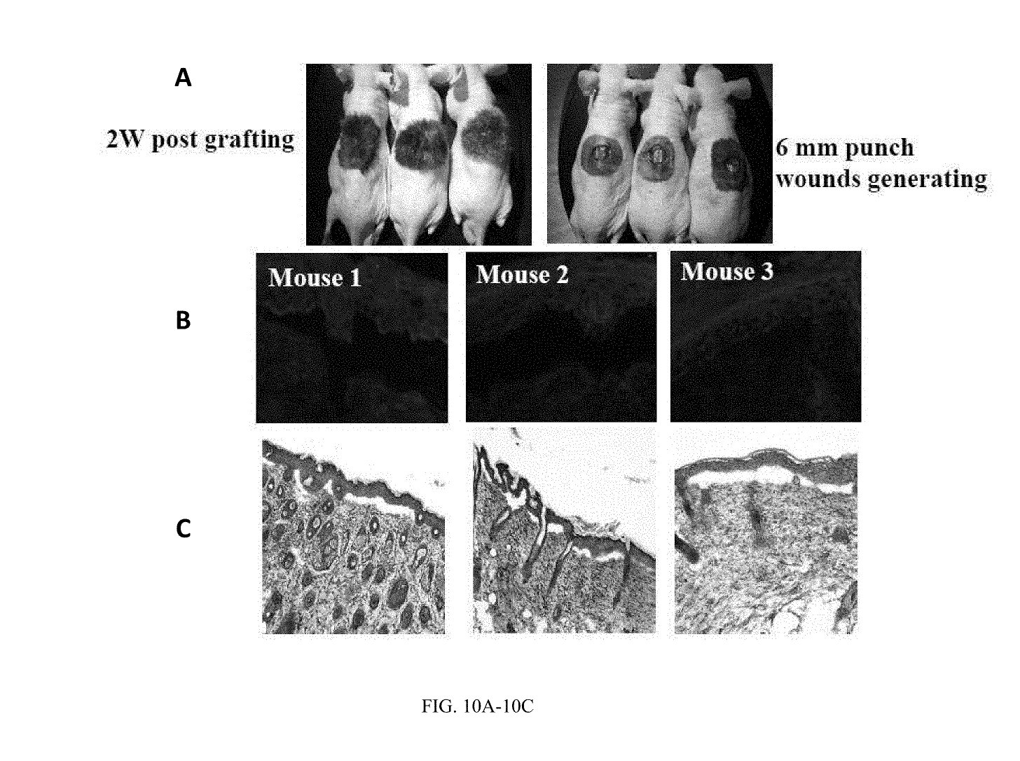

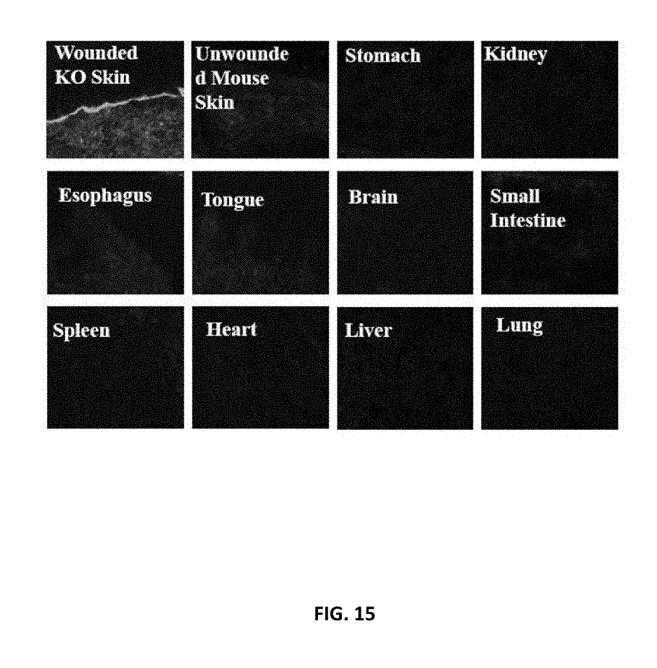

One aspect of the present invention is that C7 homes to unwounded skin in a subject with EB but not to the unwounded skin in a subject without EB. For example, In DEB skin graft model described, for instance, in FIGS. 10-14, only the grafted skin from collagen C7 knock out mice had DEB phenotype while all other organs or unwounded skin (showing in FIG. 15) were from normal host mice (i.e. a mouse without EB). In this case, the only apparent affect of IV rC7 treatment was on the DEB skin graft. The unwounded non-DEB skin and other organs from the “normal” host mice were not affected. This evidences that IV rC7 does not home to or accumulate in the unwounded skin or organs from so-called “normal” or non-EB tissue. Conversely, in the case of hypoimorphic mice of FIGS. 1-9 (“RDEB mice”), the skin as well as esophagus, tongue, small intestine were all affected. This evidences the homing of IV rC7 to even unwounded skin and tissues of the RDEB mice. Without being limited to theory, it is suggested that IV rC7 homes to the portions of the skin or other tissue of the EB patient sufficiently affected by reduced levels of C7 in EB affected tissues even when such effects do not manifest as visible wounds.

Dystrophic epidermolysis bullosa can be classified into three major types. Although the types differ in severity, their features overlap significantly and they are caused by mutations in the COL7A1 collagen 7, which encodes the protein collagen 7. Collagen 7 functions to strengthen and stabilize the skin, and is a major component of anchoring fibrils, which help anchor the top layer of the skin, the epidermis, to the underlying dermis.

Recessive dystrophic epidermolysis bullosa (RDEB) is the most severe and classical form of the disease, and is characterized by extensive blistering and scarring of the skin and mucosal membranes. The most severe form of RDEB is referred to as RDEB severe generalized (formerly known as Hallipeau-Siemens type RDEB). The COL7A1 mutations associated with RDEB impair the ability of collagen 7 to form anchoring fibrils and to connect the epidermis and dermis; and subsequent separation of the epidermis and dermis as a result of friction or minor injury causes the severe blistering and extensive scarring of the skin associated with RDEB. The onset of RDEB disease symptoms is usually at birth or early infancy, with generalized blistering presenting at birth. Subsequent extensive dystrophic scarring, most prominent on the acral surfaces, often leads to the development of pseudosyndactyly of the hands (i.e. mitten-hand deformity) and the feet during infancy. Flexion contractures of the extremities also become increasingly common with age, along with rectal and esophageal blistering.

A less severe form of autosomal recessive dystrophic epidermolysis bullosa is known as the RDEB-generalized, other or non-Hallopeau-Siemens type (non-HS RDEB). This form of the condition is somewhat less severe than the classic type and includes a range of subtypes. Blistering is limited to the hands, feet, knees, and elbows in mild cases, but may be widespread in more severe cases. Affected people often have malformed fingernails and toenails. Non-HS RDEB involves scarring in the areas where blisters occur, but this form of the condition has less severe scarring than the RDEB severe generalized type. There are other forms of RDEB, including RDEB-inversa and RDEB-pruriginosa.

Another major type of dystrophic epidermolysis bullosa is known as the autosomal dominant type (DDEB). The signs and symptoms of this condition tend to be milder than those of the autosomal recessive forms, with blistering often limited to the hands, feet, knees, and elbows. The blisters heal with scarring, but it is less severe. Most affected people have malformed fingernails and toenails, and the nails may be lost over time. In the mildest cases, abnormal nails are the only sign of the condition.

Indications

The disclosure features a pharmaceutical composition comprising collagen 7, or functional fragment or variant thereof, and one or more pharmaceutically acceptable carriers, for use in treating EB (e.g., DEB, e.g., RDEB or DDEB). The disclosure features a pharmaceutical composition comprising collagen 7, or functional fragment or variant thereof, e.g., described herein, and one or more pharmaceutically acceptable carriers for use in preventing, preventing the progression of, or delaying the onset of one or more symptom associated with scarring, e.g., scarring associated with blisters, in subjects with EB (e.g., DEB, e.g., RDEB or DDEB).

Symptoms Associated with Scarring in Subjects with EB

EB (e.g., DEB, e.g., RDEB or DDEB) symptoms associated with scarring include, but are not limited to, contractures, e.g., flexion contractures (e.g., of the extremities); pseudosyndactyly, e.g., pseudosyndactyly of the hands and pseudosyndactyly of the feet; carcinoma (e.g., squamous cell carcinoma); rectal and anal lesions; urethral lesions; mucosal lesions; lesions of squamous epithelial tissue; lesions of the gastrointestinal tract; bulla formation; bulla formation post manual trauma; nail deformities; teeth deformities; constricted esophagus; eye disorders, anemia, malnutrition; secondary skin infection; sepsis; hoarse voice; urethral stenosis; phimosis; corneal scarring; malabsorption; and failure to thrive

### Subject Selection

Subjects who may benefit from the use of the methods described herein include, but are not limited to, subjects diagnosed with EB (e.g., DEB, e.g., RDEB or DDEB). In addition, or alternatively, the subject may have, or may be at risk of developing, an EB (e.g., DEB, e.g., RDEB or DDEB) symptom associated with scarring, including but not limited to, skin blistering; pseudosyndactyly; flexion contractures of the extremities; mucosal lesions; lesions of squamous epithelial tissue; lesions of the gastrointestinal tract; bulla formation; bulla formation post manual trauma; nail deformities; teeth deformities; constricted esophagus; eye disorders; anemia; malnutrition; secondary skin infection; sepsis; hoarse voice; urethral stenosis; phimosis; corneal scarring; malabsorption; or failure to thrive. The methods of the invention can be applied advantageously to any EB patient subpopulation, e.g., DEB, e.g., RDEB or DDEB; or those with a specific EB (e.g., DEB, e.g., RDEB or DDEB) symptom associated with scarring, e.g., of blisters, e.g., those patients with pseudosyndactyly.

The present disclosure provides for methods of selecting subjects to utilize the methods of the present invention based on directly acquiring, indirectly acquiring, or acquiring knowledge of the genotype of the COL7A1 gene of a subject. The present invention provides for methods of selecting patients based on determining or acquiring knowledge of whether a subject has EB (e.g., DEB, e.g., RDEB or DDEB); or has or may be at risk of developing an EB (e.g., DEB, e.g., RDEB or DDEB) symptom associated with scarring, e.g., scarring associated with blisters and/or lesions, including but not limited to, skin blistering; pseudosyndactyly; flexion contractures of the extremities; mucosal lesions; lesions of squamous epithelial tissue; lesions of the gastrointestinal tract; bulla formation; bulla formation post manual trauma; nail deformities; teeth deformities; constricted esophagus; eye disorders; anemia; malnutrition; secondary skin infection; sepsis; hoarse voice; urethral stenosis; phimosis; corneal scarring; malabsorption; or failure to thrive.

### Preparation of Collagen 7 and Functional Fragments and Variants Thereof

Collagen 7 and functional fragments and variants thereof can be synthesized by standard molecular biology techniques in standard cell lines, e.g., CHO, HEK293, fibroblast or keratinocyte cells. Standard cell culture procedures and conditions may be used for culture of host cells described herein and are known to those skilled in the art. Host cells cultured for expression of recombinant collagen 7, such as HEK293 cells, may be cultured in routinely used cell culture media (e.g. Dulbecco's modified Eagle's medium (DMEM)/Ham's F-12 (1:1) with suitable supplementation of serum, antibiotics, etc, dependent on the application) as referenced in, ((Chen et al. J Bio Chem 277(18): 2118-2124 (2002)), (Chen et al. J Bio Chem 275: 32(11): 24429-24435 (2000)), (Chen et al. J Bio Chem 276(24): 21649-21655 (2001)).

Host cells may be engineered to express other proteins to optimize production of the recombinant collagen 7. This may include, but not limited to, the co-expression of the processing enzymes prolyl hydroxylase, prolidase, or glycosyl-transferase, by exogenously introducing isolated nucleic acid or recombinant expression vectors encoding the appropriate nucleic acid sequence, in host cells comprising collagen 7 nucleic acid sequence or recombinant expression vector. The triple helical assembly of collagen 7 often requires hydroxylation and the presence of ascorbic acid in the host cell growth media. As demonstrated in the reference, (Chen et al. J Bio Chem 277 (18): 2118-2124 (2002)), recombinant type 7 collagen produced, recovered, and purified from HEK293 cells in the presence of ascorbic acid was secreted as an approximately 900-kDa protein, corresponding to the association of three type 7 collagen monomers (each monomer 290-kDa). Ascorbic acid may be used in the host cell culture conditions to aid in proper processing and assembling of the recombinant protein.

Suitable vectors for use herein are those that can express collagen 7, prolyl hydroxylase, prolidase, or glycosyl-transferase, or a functional portion thereof. In order to express the proteins described herein, the nucleotide sequence encoding the appropriate protein, or a functional equivalent, can be inserted into a suitable vector. A suitable vector contains the necessary and appropriate transcriptional and translational control sequences for expression of the inserted nucleic acid sequence. Standard methods, known to those skilled in the art, may be used to construct the recombinant expression vectors containing the nucleic acid sequences described herein. These methods include, but are not limited to, in vitro recombinant techniques, synthetic techniques, and in vivo recombination/genetic recombination; the choice of method depends on the nature of the specific nucleotide fragments and may be determined by persons skilled in the art.

Suitable vectors for use herein may contain an origin of replication and a restriction endonuclease sequence site. Persons skilled in the art would have knowledge of suitable origin of replication and restriction endonuclease sequences for use in the host cell. Suitable vectors for use herein may contain sequence elements to aid transcription, including, but not limited to, promoter and enhancer elements. Persons skilled in the art would have knowledge of various transcriptional control elements, including but not limited to, promoters, inducible promoters, and enhancer elements, that would be suitable in the host cell. Suitable vectors for use herein may also contain a selectable marker gene that encodes a product necessary for the host cell to grow and survive under specific conditions, aiding in the selection of host cells into which the vector has been introduced. Typical selection genes may include, but are not limited to, genes encoding a protein that confers resistance to an antibiotic, drug, or toxin (e.g., tetracycline, ampicilin, neomycin, hygromycin, etc). Persons skilled in the art would have knowledge of coding sequences for suitable selectable markers and reporter genes for use in the host cell.

Expression vectors described herein can be introduced into host cells via conventional transformation or transfection techniques. Transformation and transfection techniques include, but are not limited to, calcium phosphate or calcium chloride coprecipitation, DEAE-dextran-mediated transfection, lipofectamine, electroporation, microinjection, and viral mediated transfection (as referenced in U.S. Pat No. 6,632,637 (McGrew)). Persons skilled in the art would have knowledge of suitable transformation and transfection methods based on the host cell/vector combination. For long term, high yield production of recombinant proteins, stable expression of the recombinant protein may be preferred. Host cells that stably express the recombinant protein may be engineered.

The recombinant expression vectors described herein may be introduced into a suitable host cell, which may include a living cell capable of expressing the protein coding region from the defined recombinant expression vector. The term “host cell” refers not only to the particular subject cell but to the progeny or potential progeny of the particular subject cell. Because certain modifications may occur in succeeding generations due to either mutation or environmental influences, such progeny may not in fact, be identical to the parent cell, but are still included within the scope of the term as used herein. Various host cell expression systems may be utilized to express the nucleic acid molecules described herein. These include, but are not limited to yeast or fungi, transformed with recombinant yeast or fungi expression vectors containing the appropriate nucleic acid sequence; insect cell systems infected with recombinant virus expression vectors or transformed with recombinant plasmid expression vectors containing the appropriate nucleic acid sequence; or mammalian cell systems (e.g., primate cell, human cell, rodent cell, etc) transfected with expression vectors containing the appropriate nucleic acid sequence. Suitable host cells may include primary or transformed cell lines, including, but not limited to, fibroblasts, CHO, HEK293, C127, VERO, BHK, HeLa, COS, MDCK, etc (as referenced in U.S. Pat No. 6,632,637 (McGrew)). Other suitable host cells are known to those skilled in the art.

Modifications, including, but not limited to, glycosylation, phosphyorylation, hydroxylation, and processing of protein products may be important to the function of a protein. Different host cells have various characteristics and mechanisms for post-translational processing and modification of proteins. A host cell that is capable of modulating expression of the nucleic acid sequences contained in the vector, or modulating expression of the vector nucleic acid sequences, or modifying and processing the gene product encoded in the vector sequence in a specific manner may be chosen. Mammalian host cells may be chosen to ensure the correct modification and processing of the recombinant protein. Such mammalian host cells may include, but are not limited to, CHO, HEK293, human fibroblasts, and human keratinocytes.

Proteins produced by recombinant methods described herein may be recovered from the host cell culture system according to standard protocols known in the art (e.g., precipitation, centrifugation, etc). Recombinant collagen 7 described herein may be secreted into the host cell medium and recovered by ammonium sulfate precipitation and subsequent centrifugation; as demonstrated in the following reference, (Chen et al. J Bio Chem 277(18): 2118-2124 (2002)). Proteins produced and recovered by recombinant and molecular biology methods described herein, may be purified according to standard protocols known in the art (e.g., dialysis, stepwise salt solubilization, ion exchange chromatography, affinity chromatography, SDS gel electrophoresis, etc). The recombinant collagen 7 described herein may be purified to homogeneity by ion exchange chromatography; as demonstrated in the following reference, (Chen et al. J Bio Chem 277(18): 2118-2124 (2002)).

Optionally collagen 7 may be further purified. Purification may be achieved using any method known in the art, including, but not limited to affinity chromatography, e.g., an anti-collagen 7 antibody column; hydrophobic interaction chromatography; ion exchange chromatography; size exclusion chromatography; electrophoretic procedures, e.g., isoelectric focusing, differential solubility (e.g., ammonium sulfate precipitation), or extraction, and the like.

### Compositions

The disclosure provides a pharmaceutical composition comprising collagen 7 or functional fragment or variant thereof. Pharmaceutical compositions may take the form of any acceptable pharmaceutical formulation. Pharmaceutical compositions can be formulated in a variety of different forms, such as liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application.

Exemplary pharmaceutical compositions are described below. The pharmaceutical compositions include those suitable for parenteral (including intravenous, subcutaneous, intradermal, intramuscular, and intraarticular), topical (including dermal, transdermal, transmucosal, buccal, sublingual, and intraocular), and rectal administration, although the most suitable route may depend upon, for example, the condition and disorder of the recipient.

Compositions for parenteral administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The composition may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described. Exemplary compositions for parenteral administration include injectable solutions or suspensions which can contain, for example, suitable non-toxic, parenterally acceptable diluents or solvents, such as EDTA, mannitol, 1,3-butanediol, water, Ringer's solution, an isotonic sodium chloride solution, or other suitable dispersing or wetting and suspending agents. The compositions may contain pharmaceutically acceptable substances or adjuvants, including, but not limited to, EDTA, e.g., 0.5 mM EDTA; pH adjusting and buffering agents and/or tonicity adjusting agents, e.g., sodium acetate, sodium lactate, sodium chloride, potassium chloride, calcium chloride, sorbitan monolaurate; minor amounts of non-toxic auxiliary substances, such as wetting or emulsifying agents or preservatives.

It should be understood that in addition to the ingredients particularly mentioned above, the composition may include other agents conventional in the art having regard to the type of formulation in question.

### Chronic Administration Regimen

In practicing the methods described herein, collagen 7 or functional fragment or variant thereof may be chronically administered. Chronic administration can include the administration of more than one dose of an agent over a period of time. Chronic administration can include regular administration for an extended period of time. Chronic administration can include the administration of therapy over a prolonged period of time, in some cases, for the duration of a subject's lifetime, so that the concentration of the therapeutic agent is maintained at a therapeutically or prophylactically effective level throughout the course of treatment.

The period of time of chronic administration can be for the lifetime of the subject. The period of time of chronic administration can include, but is not limited to, at least 3 months, at least 6 months, at least 1 year, at least 2 years, at least 3 years, at least 4 years, at least 5 years, at least 10 years, at least 15 years, at least 20 years, at least 25 years, at least 30 years, at least 35 years, at least 40 years, at least 45 years, at least 50 years, at least 100 years, or anytime period between 3 months and 100 years.

Chronic administration can include a series of doses which together provide an effective amount for treating EB (e.g., DEB, e.g., RDEB or DDEB); and/or preventing, preventing the progression of, or delaying the onset of EB (e.g., DEB, e.g., RDEB or DDEB) symptoms associated with scarring. A pharmaceutical composition comprising collagen 7 or functional fragment or variant thereof may be administered on various dosing schedules. The dosing schedule can be dependent on several factors including, the severity of EB (e.g., DEB, e.g., RDEB or DDEB); presence of EB (e.g., DEB, e.g., RDEB or DDEB) symptoms associated with scarring, e.g., of blisters;; the specific composition of collagen 7 or functional fragment or variant thereof employed for treatment; the age, body weight, general health, sex and diet of the patient; the time of administration, route of administration, and rate of excretion of the specific composition employed; the duration of the treatment; drugs used in combination or coincidental with the specific collagen 7 or functional fragment or variant thereof composition employed; and like factors well known in the medical arts.

Exemplary dosing schedules of collagen 7 or functional fragment or variant thereof include, once daily, or once weekly, or once every other week, or once monthly, or once every other month, or once every three months, or once every 6 months, or once every 12 months, or once every 18 months, or once every 24 months. The composition can be administered twice per week or twice per month, or once every two, three or four weeks. The composition can be administered as two, three, or more sub-doses at appropriate intervals or even using continuous infusion or delivery through a controlled release formulation. In that case, the therapeutic agent contained in each sub-dose may be correspondingly smaller in order to achieve the total daily dosage. The dosage can also be compounded for delivery over several days, e.g., using a conventional sustained release formulation, which provides sustained release of the agent over a several day period. Sustained release formulations are well known in the art and are particularly useful for delivery of agents at a particular site. The total daily, weekly, or monthly usage of collagen 7 composition can be decided by an attending physician within the scope of sound medical judgment.

The present disclosure features methods including, administering a treatment comprising collagen 7 or functional fragment or variant thereof. The disclosure can further include selecting a regimen, e.g., dosage, formulation, route of administration, number of dosages, or adjunctive or combination treatments of collagen 7 or functional fragment or variant thereof. The disclosure can further include the administration of the selected regimen. The administration can be based on knowledge of an EB (e.g., DEB, e.g., RDEB or DDEB) patient possessing an EB (e.g., DEB, e.g., RDEB or DDEB) symptom associated with scarring, e.g., of blisters, or a plurality of EB (e.g., DEB, e.g., RDEB or DDEB) symptoms associated with scarring, e.g., of blisters, as described herein.

Even upon improvement of a patient's condition, chronic administration of collagen 7 or functional fragment or variant thereof may be administered. The dosage or frequency of administration, or both, may not be reduced, as a function of EB (e.g., DEB, e.g., RDEB or DDEB) symptoms. The dosage or frequency of administration, or both, may be reduced, as a function of EB (e.g., DEB, e.g., RDEB or DDEB) symptoms, to a level at which the improved condition is retained. Subjects may require intermittent changes in dosage or frequency of administration of treatment on a long-term basis upon any recurrence of EB (e.g., DEB, e.g., RDEB or DDEB) symptoms.

### Combination Treatments

The present disclosure encompasses combined administration of an additional agent with collagen 7 or functional fragment or variant thereof. An additional agent may include, but is not limited to, antibiotics, analgesics, opioids, anti-virals, anti-inflammatory agents, or nutritional supplements.

Antibiotics can include, but are not limited to, Aknilox, Ambisome, Amoxycillin, Ampicillin, Augmentin, Avelox, Azithromycin, Bactroban, Betadine, Betnovate, Blephamide, cancidas, Cefaclor, Cefadroxil, Cefdinir, Cefepime, Ceflx, Cefixime, Cefoxitin, Cefpodoxime, Cefprozil, Cefuroxime, Cefzil, Cephalexin, Cephazolin, Ceptaz, Chloramphenicol, Chlorhexidine, Chloromycetin, Chlorsig, Ciprofloxacin, Clarithromycin, Clindagel, Clindamycin, Clindatech, Cloxacillin, Colistin, Co-trimoxazole, Demeclocycline, Diclocil, Dicloxacillin, Doxycycline, Duricef, Erythromycin, Flagyl alcohol, Flagyl dosage, Flagyl pregnancy, Flagyl side effects, Flagyl treatment, Flamazine, Floxin, Framycetin, Fucidin, Furadantin, Fusidic, Gatifloxacin, Gemifloxacin, Gemifloxacin, Ilosone, Iodine, Levaquin, Levofloxacin, loceryl, Lomefloxacin, Maxaquin, Mefoxin, Meronem, Minocycline, Moxifloxacin, Myambutol, Mycostatin, Neosporin, Netromycin, Nitrofurantoin, Norfloxacin, Norilet, Ofloxacin, Omnicef, Ospamox, Oxytetracycline, Paraxin, Penicillin, Pneumovax, Polyfax, Povidone, Rifadin, Rifampin, Rifaximin, Rifmah, Rimactane, Rocephin, Roxithromycin, Seromycin, Soframycin, Sparfloxacin, Staphlex, Targocid, Tetracycline, Tetradox, Tetralysal, tobramycin, Tobramycin, Trecator, Tygacil, Vancocin, Velosef, Vibramycin, Xifaxan, Zagam, Zitrotek, Zoderm, Zymar, and Zyvox.

Anti-virals include, but are not limited to, Abacavir, Aciclovir, Acyclovir, Adefovir, Amantadine, Amprenavir, Ampligen, Arbidol, Atazanavir, Atripla,Boceprevir, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir,Idoxuridine, Imiquimod, Indinavir, Inosine, Integrase inhibitor, Interferon type III, Interferon type II, Interferon type I, Interferon, Lamivudine, Lopinavir, Loviride, Maraviroc, Moroxydine, Methisazone, Nelfmavir, Nevirapine, Nexavir, Nucleoside analogues, Oseltamivir, Peginterferon alfa-2a, Penciclovir, Peramivir, PleconarilPodophyllotoxin, Protease inhibitor, Raltegravir, Reverse transcriptase inhibitor, Ribavirin, Rimantadine, Ritonavir, Pyramidine, Saquinavir, Stavudine, Tea tree oil, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir , and Zidovudine.

Anti-inflammatory agents include, but are not limited to, Ibuprofen, Naproxen, Aspirin, Diclofenac, Indomethacin, Ketoprofen, Piroxicam, Meloxicam, Sulindac, and Steroids, Non-steroidal anti-inflammatory agents, e.g., aspirin, ibuprofen, and naproxen.

Nutritional supplements include, but are not limited to, iron, calcium, vitamin D, selenium, carnitine, and zinc.

When collagen 7 or functional fragment or variant thereof is administered in combination with an additional agent or a plurality of agents, the dosage of collagen 7 or functional fragment or variant thereof may on its own comprise an effective amount and an additional agent or agents may further augment the therapeutic benefit to the patient. Alternatively the combination of collagen 7 or functional fragment or variant thereof and a second agent may together comprise an effective amount for treating EB (e.g., DEB, e.g., RDEB) and/or preventing, preventing the progression of, or delaying the onset of EB (e.g., DEB, e.g., RDEB) symptoms associated with scarring, e.g., of blisters. The effective amounts may be defined in the context of particular treatment regimens, including, e.g., timing and number of administrations, modes of administrations, formulations, etc.

### Treatment Outcomes

The present disclosure provides methods of treating EB (e.g., DEB, e.g., RDEB or DDEB) and/or preventing, preventing the progression of, or delaying the onset of EB (e.g., DEB, e.g., RDEB or DDEB) symptoms associated with scarring, e.g., of blisters. Symptoms associated with scarring of blisters can include, but are not limited to, skin blistering; contractures, e.g., flexion contractures (e.g., of the extremities); pseudosyndactyly, e.g., pseudosyndactyly of the hands and pseudosyndactyly of the feet; carcinoma (e.g., squamous cell carcinoma); rectal, anal, and urethral lesions; mucosal lesions; lesions of squamous epithelial tissue; lesions of the gastrointestinal tract; bulla formation; bulla formation post manual trauma; nail deformities; teeth deformities; constricted esophagus; eye disorders, anemia, malnutrition; secondary skin infection; sepsis; hoarse voice; urethral stenosis; phimosis; corneal scarring; malabsorption; and failure to thrive.

The severity of EB (e.g., DEB, e.g., RDEB or DDEB) and symptoms associated with scarring of blisters may be assessed using conventional clinical methods known in the medical arts, and include but are not limited to, measure of digital motor ability, level of pseudosyndactyly of the hands and feet, number and severity of oral lesions, number and severity of rectal lesions, body weight, nutritional deficiencies, presence of secondary infections, etc.

Assessments may be performed at least 14 days from the start of treatment according to the invention, e.g., at least about 7 days or at least about 10 days from the start of treatment. Assessments may be performed at regularly scheduled times post the start of treatment, e.g., every week, every 2 weeks, every 3 weeks, every 4 weeks, every month, every other month, every 3 months, every 6 months, post the start of treatment. Assessments pay be performed at least 7 days after each dose where multiple doses are administered, e.g., at least 7 days after each dose, or 14 days after each dose. Assessments may be performed at regularly scheduled times post each dose when multiple doses are administered, e.g., every week, every 2 weeks, every 3 weeks, every 4 weeks, every month, every other month, every 3 months, every 6 months, post each dose. Assessments may be performed at regularly scheduled times post each dose when multiple doses are administered, e.g., at the time of the next dose. The timing of assessment may be altered throughout the course of chronic administration. The timing of assessment may be altered in response to a change in presence or absence of EB (e.g., DEB, e.g., RDEB or DDEB) symptoms associated with scarring, e.g., of blisters, or a change in the severity of EB (e.g., DEB, e.g., RDEB or DDEB) symptoms associated with scarring, e.g., of blisters.

### EXEMPLIFICATION

### Example 1. RDEB Mouse Model of IV Administration of Collagen 7

RDEB (C7 hypomorphic) mice show a similar clinical phenotype to human DEB patients, including blistering, forepaw pseudosyndactyly, growth retardation, and rectal blistering. As occurs in human DEB, hypomorphic RDEB mice have been shown to express collagen 7 at about 10% of normal level at the basement membrane zone of the skin and display separation of the dermis and epidermis in the skin compared. In addition, the phenotypes of these mice closely mimic characteristics of severe human RDEB, including mucocutaneous blistering, nail dystrophy, and mitten deformities of the extremities. Thus, the RDEB mouse model provides an excellent animal model for the study of DEB.

In order to establish the use of the RDEB mouse model for IV administration of collagen 7, two RDEB mice with full thickness skin wounds were dosed with 20 μg or 200 μg of collagen 7 via tail vein IV injection. Accordingly, mice were given either a single IV injection of 20 μg of collagen 7 in 50 μl of PBS (phosphate buffered saline) with 0.5 mM EDTA (ethylenediaminetetraacetic acid) or two IV injections of 100 μg of collagen 7, each in 250 μl of PBS with 0.5 mM EDTA. Seven days post collagen 7 administration, the presence of collagen 7 at both the skin and tongue wounds was assessed using immunofluorescence with an monoclonal antibody specific to human collagen C7. Un-injected RDEB mice and PBS-treated mice were used as controls.

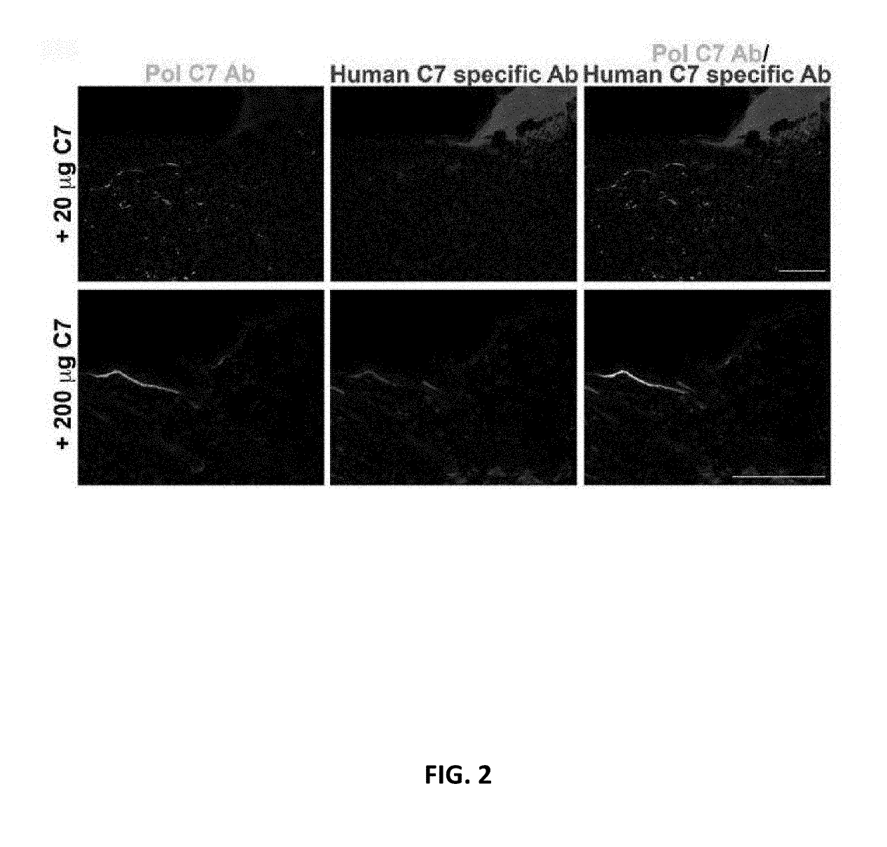

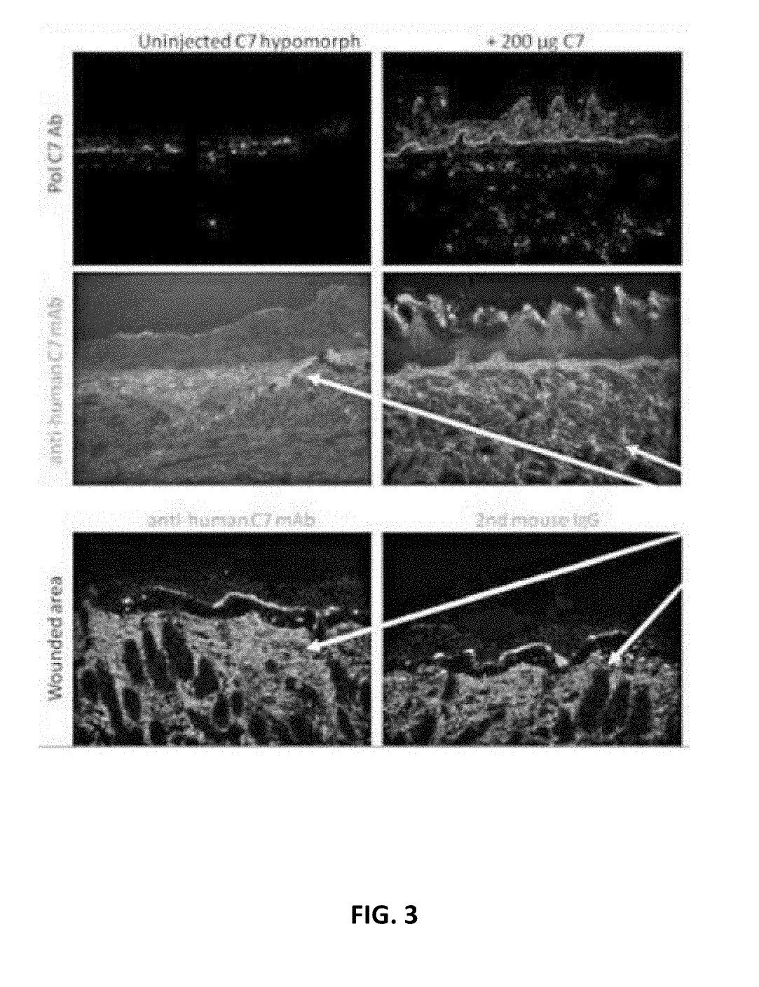

Systemically administered collagen 7 was demonstrated to home to the skin wounds, and specifically the basement membrane zone, without adverse affects in RDEB mice, as shown in FIG. 1. In addition, the systemically administered collagen 7 was shown to home to wounded areas in a dose dependent manner, with increased collagen 7 present at the skin basement membrane zone in mice injected with 200 μg of collagen 7 compared to 20 μg of collagen 7 (FIG. 2). As shown in FIG. 3, collagen 7 also homed to the wounded area of the tongue post systemic IV administration. Overall, the results demonstrate IV administered collagen 7 homes to wounded skin in a dose dependent matter, and collagen 7 can be delivered via IV injection without any adverse affects in the RDEB mouse model.

### Example 2. Chronic Collagen 7 Administration Prevents, Reduces, and Delays RDEB Associated Complications in RDEB Mice

In order to evaluate the effect of chronic collagen 7 administration on the RDEB phenotype, three RDEB mice were injected with 200 μg of collagen 7 every other week for 12 weeks or until demise. Three PBS-treated RDEB mice were used as controls. Collagen 7 was mainly administered via IV tail vein injections. In some cases collagen 7 was injected retro-orbitally when tail vein injections were difficult to perform. During weekly evaluation the mice were weighed and photographed, along with examination of their paws, skin, and rectum. At the end of the 12 week period the skin and other organs were analyzed for collagen 7 expression.

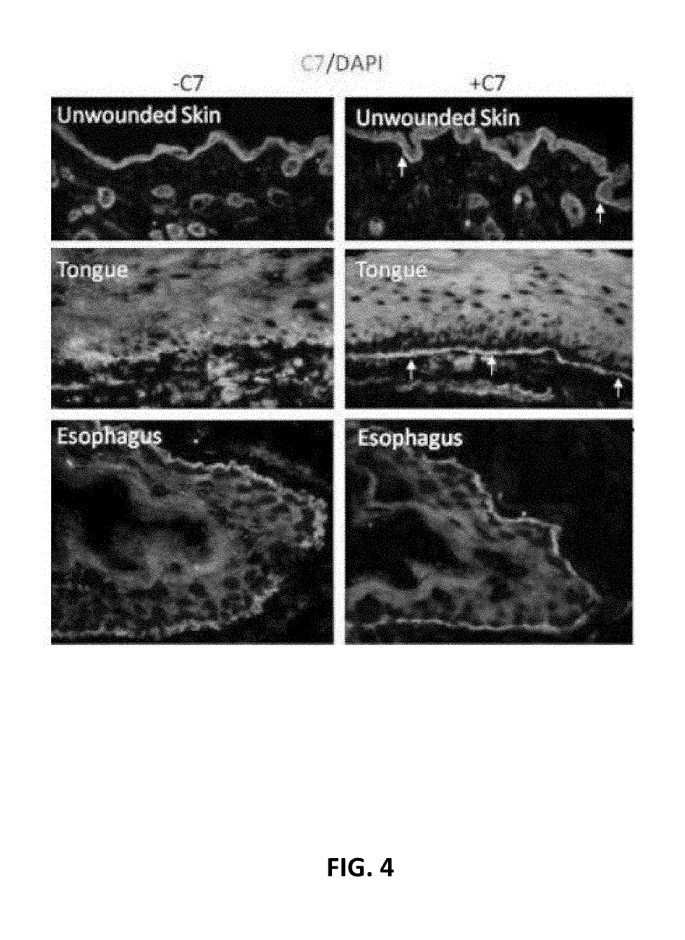

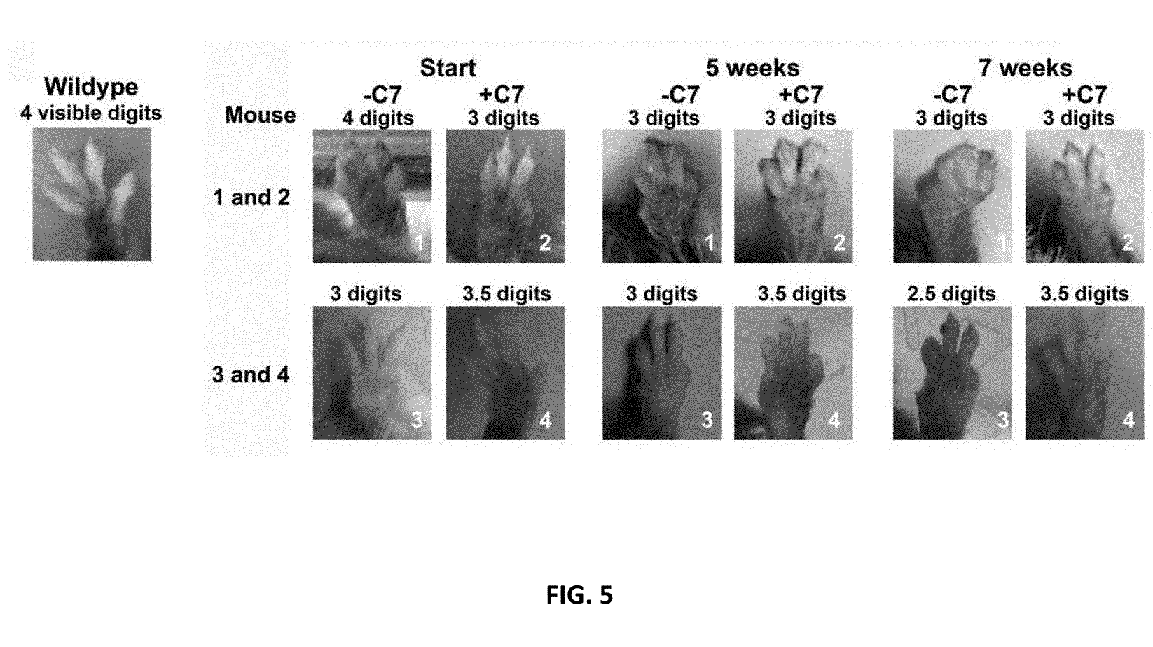

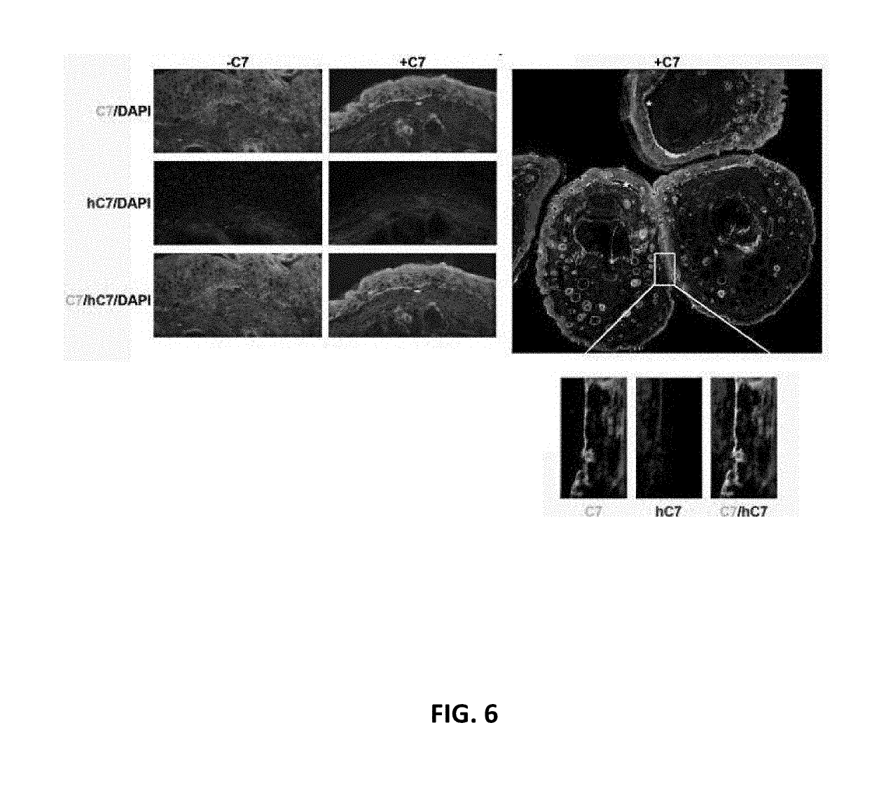

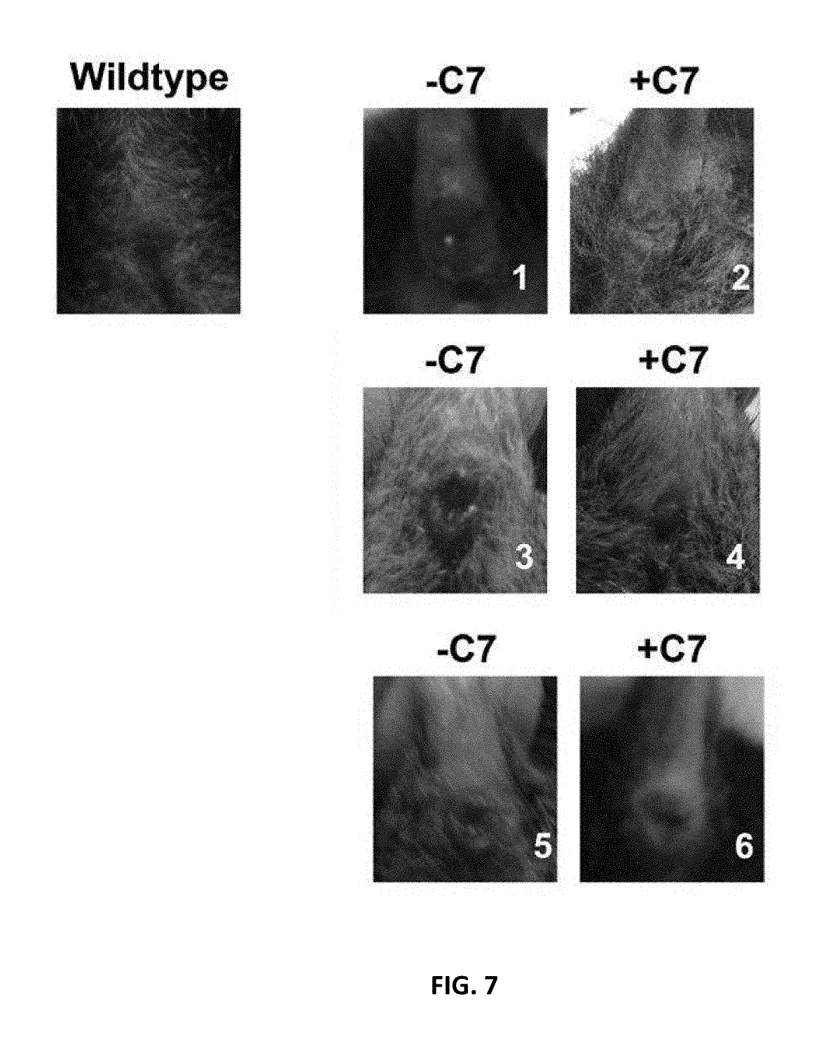

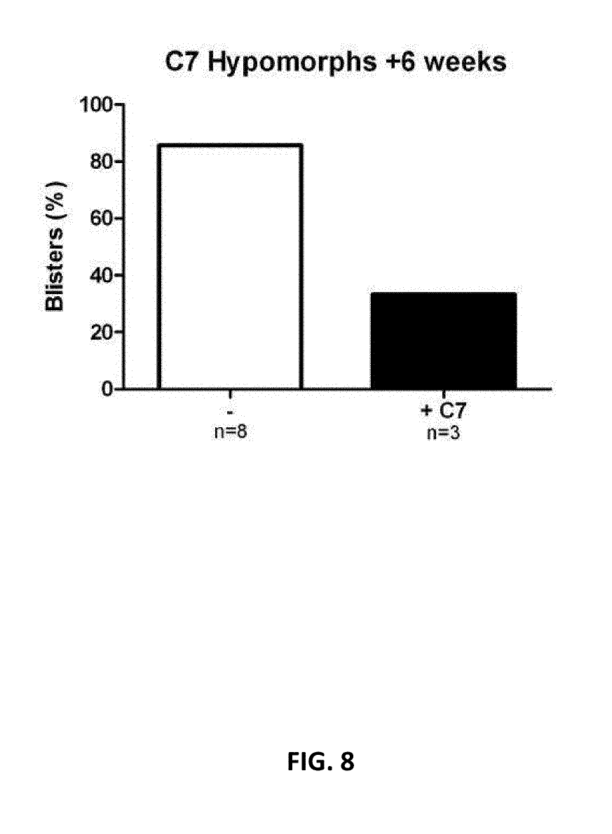

Immunofluorescence analysis demonstrated chronic administration of collagen 7 resulted in the deposition of human collagen 7 in the skin, tongue, and esophagus (FIG. 4). Moreover, chronic collagen 7 administration delayed forepaw pseudo syndactyly in RDEB mice (FIG. 5). Immunofluorescence analysis of the forepaws of collagen 7 treated mice showed deposition of collagen 7 in the digit dermal-epidermal junction at areas of high stress (FIG. 6). As shown in FIG. 7 and FIG. 8, chronic collagen 7 administration also protected against rectal blistering. There were no obvious rectal blisters in 2 out of the 3 collagen 7 treated mice, along with a greatly decreased percentage of blistering in the collagen 7 treatment group at 6 weeks post treatment initiation (FIG. 8).

Overall, the results show chronic collagen 7 administration results in the prevention, reduction, and delay of EB (e.g., DEB, e.g., RDEB) symptoms associated with scarring of blisters, resulting in a better overall EB phenotype. This is evidenced by both protection from and reduced rectal blistering, as well as the prevention of progression of and reduced forepaw pseudosyndactyly.

### Example 3. IV Administered Recombinant Collagen 7 Homes Selectively to Wounded and Unwounded DEB Skin Grafted Onto Athymic Nude Mice

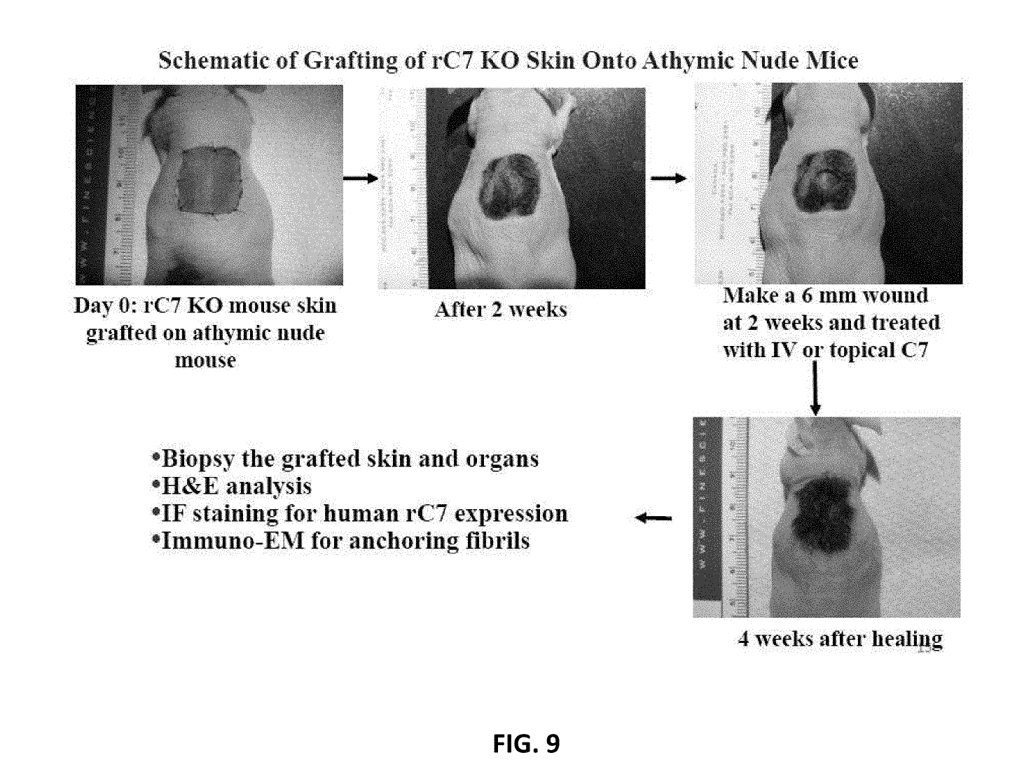

In order to evaluate the homing and deposition of recombinant collagen 7 administered intravenously, an DEB skin grafted onto athymic nude mouse model, in which the mice possess skin grafts obtained from collagen 7 knock out RDEB mice were utilized (FIG. 9). In brief, skin from collagen 7 knockout RDEB mice that completely lack collagen C7 expression at the basement membrane zone was grafted onto athymic nude mice. After two weeks, the RDEB skin was wounded using 6 mm punch biopsies or left unwounded and recombinant collagen 7 was administered IV via tail vein injection. The mice were sacrificed at two to three weeks post collagen 7 administration, and skin biopsies from the grafted DEB skin were analyzed by hematoxylin and eosin stain, immunofluorescence, or electron microscopy. Biopsies from various other organs from the host athymic nude mice were taken at two and three weeks post collagen 7 administration as well, and analyzed by immunofluorescence for recombinant human collagen 7 deposition.

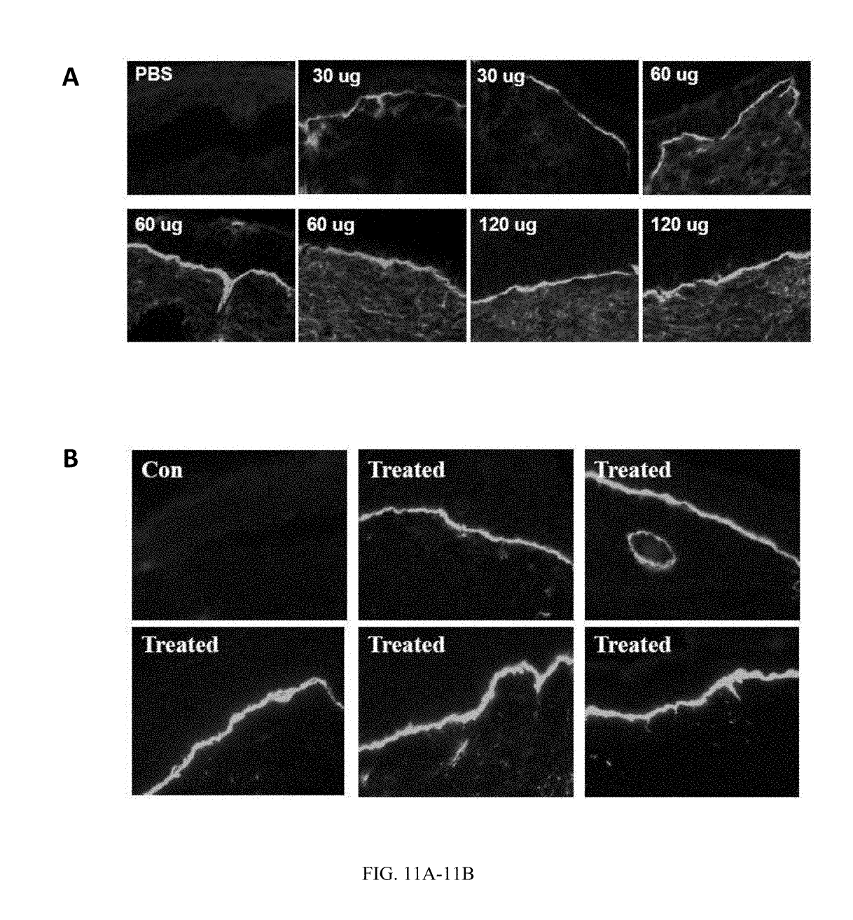

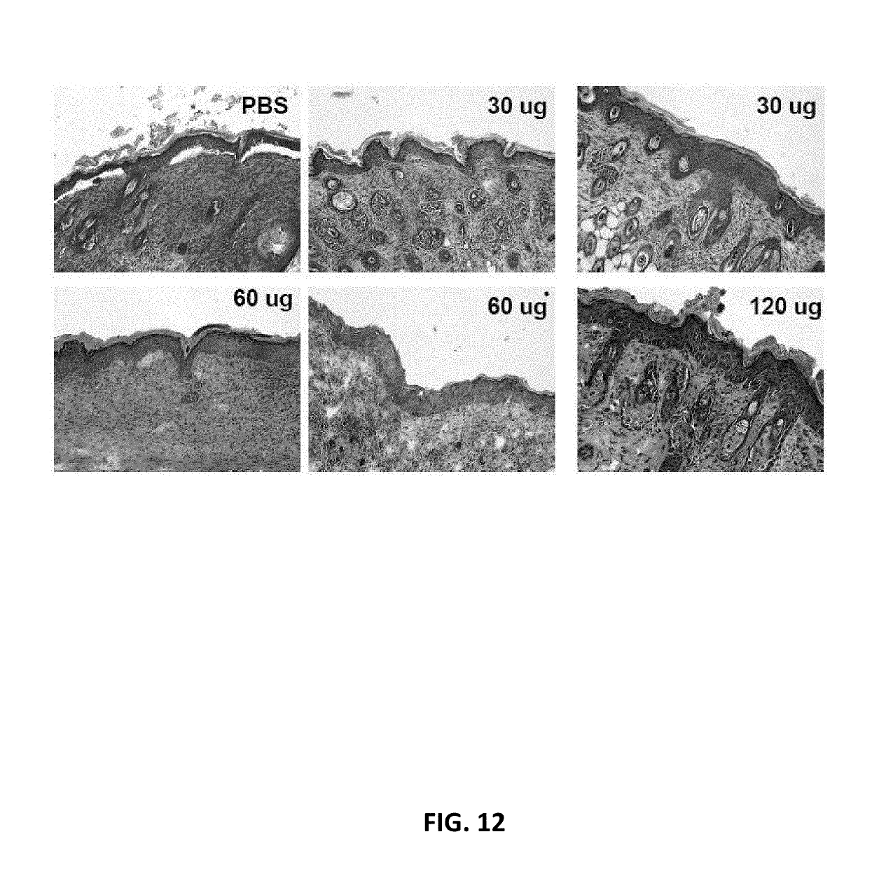

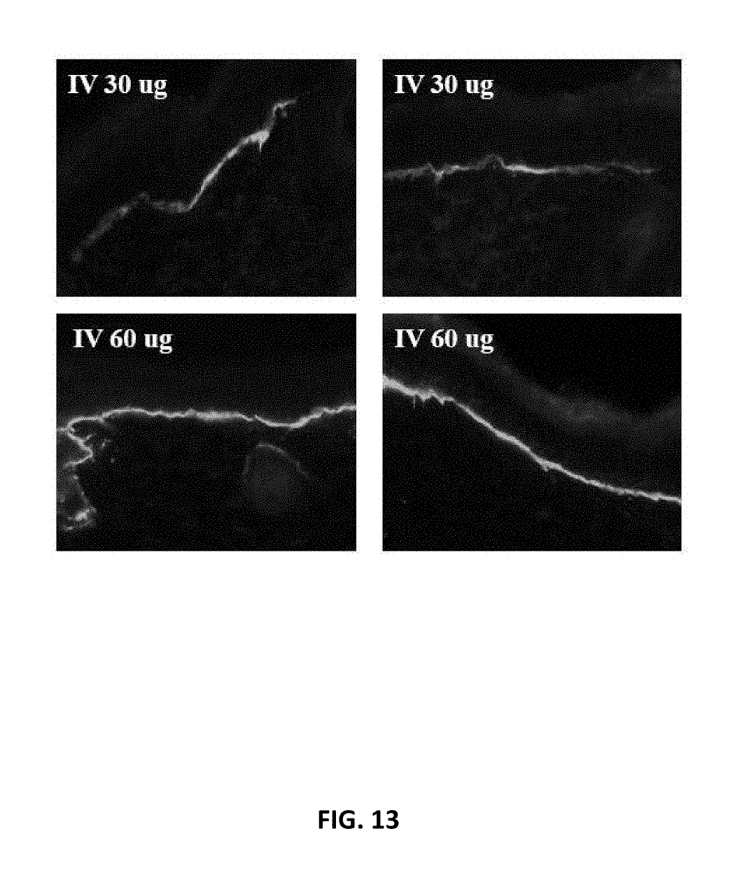

To establish the use of the above described mouse model for analysis of IV administration of collagen 7, the phenotype of the grafted RDEB skin along with collagen 7 expression was first analyzed. As shown in FIG. 10, the grafted skin retained the RDEB phenotype, with immunofluorescence (FIG. 10B) and hematoxylin and eosin staining (FIG. 10C) conducted two weeks post skin grafting (using the anti-collagen 7 NC1 domain pAb), showing no collagen 7 expression and dermal-epidemal separation. When collagen 7 was administered (IV via tail vein injection), collagen 7 expression was restored in the basement membrane zone of the wounded RDEB skin grafts, with immunofluorescence showing recombinant collagen 7 expression in the basement membrane zone of the wounded RDEB skin 2-3 weeks post collagen 7 IV administration (FIG. 11A and FIG. 11B), along with correction of the dermal-epidermal separation seen in the collagen 7 knock out wounded skin grafts (FIG. 12). In addition, immunofluorescence analysis showed a dose dependent deposition of recombinant collagen 7 at the basement membrane zone of the wounded RDEB skin grafts (FIG. 13).

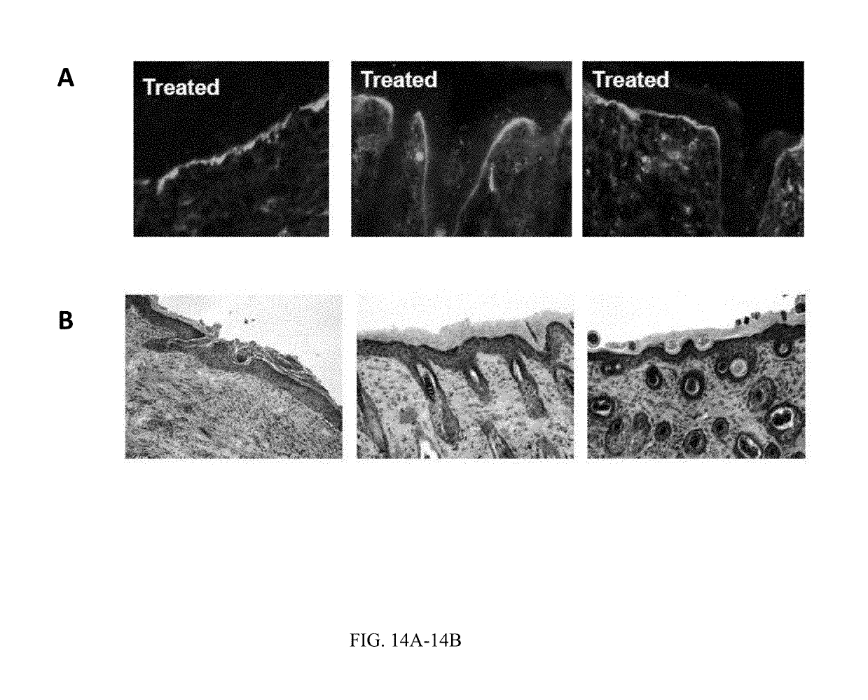

Surprisingly, analysis of unwounded RDEB skin grafts showed that even when no wounds were made to the grafted RDEB skin, IV administered collagen 7 homed to and incorporated into the basement membrane zone of the unwounded RDEB skin (FIG. 14). Sixty μg of recombinant collagen 7 was delivered IV via tail vein injection to unwounded RDEB skin grafted onto athymic nude mice. When samples of the skin from unwounded RDEB skin grafts were analyzed, immunofluorescence (FIG. 14A) and hematoxylin and eosin staining (FIG. 14B) showed the restoration of collagen 7 expression in the unwounded skin and correction of dermal-epidermal separation. Therefore, we conclude that IV recombinant collagen 7 homes to and incorporates in not only wounded skin but also unwounded skin as well.

In order to further analyze the homing and deposition of IV administered collagen 7, biopsies from various organs, including the stomach, kidney, esophagus, tongue, brain, small intestine, spleen, heart, liver, and lung from unwounded host athymic nude mice with collagen 7 knockout skin grafts and were analyzed for collagen 7 expression. For these experiments, RDEB skin grafts were first wounded and then 60-120 μg collagen 7 was delivered IV via tail vein injection to the athymic nude mice. Biopsies from wounded RDEB skin grafts and unwounded host athymic nude mouse skin or organ samples were obtained 2-3 weeks post collagen 7 administration and subjected to immunofluorescence staining using a monoclonal antibody specific to human collagen C7. Human collagen C7 expression was only detected in wounded RDEB skin grafts but not in unwounded normal skin or all organs from host athymic nude mice including the stomach, kidney, brain, spleen, heart, liver, esophagus, tongue, small intestine or lung (FIG. 15 This data shows, IV administered recombinant collagen 7 selectively homes to and incorporates into the wounded RDEB skin grafts, but not to unwounded normal mouse skin and other organs.

| | |
|---|---|
| Sequence Listing: | Collagen alpha-1(VII) chain precursor [Homo sapien] |
| NCBI Reference: | Sequence: NP_000085.1 |

The NCBI reference Sequence: NP_000085.1 an related information is hereby
incorporated by reference in its entirety.

| | |
|---|---|
| 1 | mtlrllvaal cagilaeapr vraqhrervt ctrlyaadiv flldgsssig rsnfrevrsf |
| 61 | leglvlpfsg aasaqgvrfa tvqysddprt efgldalgsg gdvirairel sykggntrtg |
| 121 | aailhvadhv flpqlarpgv pkvcilitdg ksqdlvdtaa qrlkgqgvkl favgiknadp |
| 181 | eelkrvasqp tsdffffvnd fsilrtllpl vsrrvcttag gvpvtrppdd stsaprdlvl |
| 241 | sepssqslrv qwtaasgpvt gykvqytplt glgqplpser qevnvpaget svrlrglrpl |
| 301 | teyqvtvial yansigeays gtarttaleg peltiqntta hsllvawrsv pgatgyrvtw |
| 361 | rvlsggptqq qelgpgqgsv llrdlepgtd yevtvstlfg rsvgpatshn artdasveqt |
| 421 | lrpvilgpts illswnlvpe argyrlewrr etgleppqkv vlpsdvtryq ldglqpgtey |
| 481 | rltlytlleg hevatpatvv ptgpelpvsp vtdlqatelp gqrvrvswsp vpgatqyrii |
| 541 | vrstqgvert lvlpgsqtaf dlddvqagls ytvrvsarvg pregsasvlt vrrepetpla |
| 601 | vpglrvvvsd atrvrvawgp vpgasgfris wstgsgpess qtlppdstat ditglqpgtt |
| 661 | yqvaysvlrg reegpaaviv artdplgpvr tvhvtqasss svtitwtrvp gatgyrvswh |
| 721 | sahgpeksql vsgeatvael dglepdteyt vhvrahvagv dgppasvvvr tapepvgrvs |
| 781 | rlqilnassd vlritwvgvt gatayrlawg rseggpmrhq ilpgntdsae irgleggvsy |
| 841 | svrvtalvgd regtpvsivv ttppeappal gtlhwqrge hslrlrwepv praqgfllhvv |
| 901 | qpeggqeqsr vlgpelssyh ldglepatqy rvrlsvlgpa gegpsaevta rtesprvpsi |
| 961 | elrvvdtsid svtlawtpvs rassyilswr plrgpgqevp gspqtlpgis ssqrvtglep |
| 1021 | gvsyifsltp vldgvrgpea svtqtpvcpr gladvvflph atqdnahrae atrrvlerlv |
| 1081 | lalgplgpqa vqvgllsysh rpsplfping shdlgiilqr irdmpymdps gnnlgtavvt |
| 1141 | ahrymlapda pgrrqhvpgv mvllvdeplr gdifspirea qasglnvvml gmagadpeql |
| 1201 | rrlapgmdsv qtffavddgp sldqaysgla talcqasflt qprpepcpvy cpkgqkgepg |
| 1261 | emglrgqvgp pgdpglpgrt gapgpqgppg satakgergf pgadgrpgsp gragnpgtpg |
| 1321 | apglkgspgl pgprgdpger gprgpkgepg apgqviggeg pglpgrkgdp gpsgppgprg |
| 1381 | plgdpgprgp pglpgtamkg dkgdrgergp pgpgeggiap gepglpglpg spgpqgpvgp |
| 1441 | pgkkgekgds edgapglpgq pgspgeqgpr gppgaigpkg drgfpgplge agekgergpp |
| 1501 | gpagsrglpg vagrpgakgp egppgptgrq gekgepgrpg dpavvgpava gpkgekgdvg |
| 1561 | pagprgatgv qgergppglv lpgdpgpkgd pgdrgpiglt gragppgdsg ppgekgdpgr |
| 1621 | pgppgpvgpr grdgevgekg degppgdpgl pgkagerglr gapgvrgpvg ekgdqgdpge |
| 1681 | dgrngspgss gpkgdrgepg ppgppgrlvd tgpgarekge pgdrgqegpr gpkgdpglpg |
| 1741 | apgergiegf rgppgpqgdp gvrgpagekg drgppgldgr sgldgkpgaa gpsgpngaag |
| 1801 | kagdpgrdgl pglrgeqglp gpsgppglpg kpgedgkpgl ngkngepgdp gedgrkgekg |
| 1861 | dsgasgregr dgpkgergap gilgpqgppg lpgpvgppgq gfpgvpggtg pkgdrgetgs |
| 1921 | kgeqglpger glrgepgsvp nvdrlletag ikasalreiv etwdessgsf lpvperrrgp |
| 1981 | kgdsgeqgpp gkegpigfpg erglkgdrgd pgpqgppgla lgergppgps glagepgkpg |
| 2041 | ipglpgragg vgeagrpger gergekgerg eqgrdgppgl pgtpgppgpp gpkvsvdepg |
| 2101 | pglsgeqgpp glkgakgepg sngdqgpkgd rgvpgikgdr gepgprgqdg npglpgergm |
| 2161 | agpegkpglq gprgppgpvg ghgdpgppga pglagpagpq gpsglkgepg etgppgrglt |
| 2221 | gptgavglpg ppgpsglvgp qgspglpgqv getgkpgapg rdgasgkdgd rgspgvpgsp |
| 2281 | glpgpvgpkg epgptgapgq avvglpgakg ekgapgglag dlvgepgakg drglpgprge |
| 2341 | kgeagragep gdpgedgqkg apgpkgfkgd pgvgvpgspg ppgppgvkgd lglpglpgap |
| 2401 | gvvgfpgqtg prgemgqpgp sgerglagpp gregipgplg ppgppgsvgp pgasglkgdk |
| 2461 | gdpgvglpgp rgergepgir gedgrpgqeg prgltgppgs rgergekgdv gsaglkgdkg |
| 2521 | dsavilgppg prgakgdmge rgprgldgdk gprgdngdpg dkgskgepgd kgsaglpglr |
| 2581 | gllgpqgqpg aagipgdpgs pgkdgvpgir gekgdvgfmg prglkgergv kgacgldgek |
| 2641 | gdkgeagppg rpglaghkge mgepgvpgqs gapgkeglig pkgdrgfdgq pgpkgdqgek |
| 2701 | gergtpgigg fpgpsgndgs agppgppgsv gprgpeglqg qkgergppge rvvgapgvpg |
| 2761 | apgergeqgr pgpagprgek geaalteddi rgfvrqemsq hcacqgqfia sgsrplpsya |
| 2821 | adtagsqlha vpvlrvshae eeervppedd eyseyseysv eeyqdpeapw dsddpcslpl |
| 2881 | degsctaytl rwyhravtgs teachpfvyg gcggnanrfg treacerrcp prvvqsqgtg |
| 2941 | taqd |

What is claimed is:

1. A method of treating EB comprising:
systemically administering to a subject having EB or at risk of having EB collagen 7, or functional fragments and variants thereof; and
administering subsequent doses of collagen 7, or the functional fragment or variant thereof,
such that the subject is administered collagen 7, or the functional fragment or variant thereof, over a period of at least 6 months, 9 months, 12 months, 15 months, 18 months, 21 months, 2 years, three years, four years, five years, six years or more.

2. The method of claim 1, wherein collagen 7 is administered over the lifetime of the subject.

3. The method of claim 1, wherein collagen 7 is administered once every month, 2 months, 3 months, 4 months, 5 months or 6 months.

4. The method of claim 1, wherein each subsequent collagen 7 dose is administered two weeks, three weeks, four weeks, five weeks, six weeks, or eight weeks after the previous dose.

5. The method of claim 1, wherein each subsequent collagen 7 dose is administered one month after the previous dose of collagen 7, or the functional fragment or variant thereof.

6. The method of claim 1, wherein collagen 7 is administered once every month, 2 months, 3 months, 4 months, 5 months or 6 months.

7. The method of claim 1, wherein said collagen 7 is administered intravenously.

8. The method of claim 1, wherein said subject is less than 24, less than 12, less than 6, less than 5, less than 4, less than 3, less than 2, or less than 1 month of age.

9. The method of claim 1, comprising acquiring the C7 genotype of said subject.

10. A method of preventing, preventing the progression of, or delaying the onset of one or more symptom associated with scarring of blisters in subjects with EB comprising:
systemically administering collagen 7, or a functional fragment or variant thereof, to a subject having EB, or at risk of having EB; and
administering subsequent doses of collagen 7, or the functional fragment or variant thereof, such that the subject is administered collagen 7, or the functional fragment or variant thereof, over a period of at least 6 months, 9 months, 12 months, 15 months, 18 months, 21 months, 2 years, three years, four years, five years, six years or more.

11. The method of claim 10, wherein collagen 7 is administered over the lifetime of the subject.

12. The method of claim 10, wherein collagen 7 is administered once every month, 2 months, 3 months, 4 months, 5 months or 6 months.

13. The method of claim 10, wherein each subsequent collagen 7 dose is administered two weeks, three weeks, four weeks, five weeks, six weeks, or eight weeks after the previous dose.

14. The method of claim 10, wherein each subsequent collagen 7 dose is administered one month after the previous dose of collagen 7, or the functional fragment or variant thereof.

15. The method of claim 10, wherein collagen 7 is administered once every month, 2 months, 3 months, 4 months, 5 months or 6 months.

16. The method of claim 10, wherein said collagen 7 is administered intravenously.

17. The method of claim 10, wherein said subject is less than 24, less than 12, less than 6, less than 5, less than 4, less than 3, less than 2, or less than 1 month of age.

18. The method of claim 10, comprising acquiring the C7 genotype of said subject.
